(19) United States
(12) Patent Application Publication
(10) Pub. No.: US 2019/0031999 A1
(43) Pub. Date: Jan. 31, 2019

(54) THREE-DIMENSIONAL ACOUSTIC MANIPULATION OF CELLS

(71) Applicants: Carnegie Mellon University, Pittsburgh, PA (US); The Penn State Research Foundation, University Park, PA (US); Massachusetts Institute of Technology, Cambridge, MA (US)

(72) Inventors: Subra Suresh, Pittsburgh, PA (US); Feng Guo, State College, PA (US); Zhangming Mao, State College, PA (US); Ming Dao, West Roxbury, MA (US); Tony Jun Huang, West Roxbury, PA (US)

(73) Assignees: Carnegie Mellon University, Pittsburgh, PA (US); The Penn State Research Foundation, University Park, PA (US); Massachusetts Institute of Technology, Cambridge, MA (US)

(21) Appl. No.: 16/071,641

(22) PCT Filed: Jan. 20, 2017
(86) PCT No.: PCT/US17/14346

§ 371 Date: Jul. 20, 2018

Related U.S. Application Data
(60) Provisional application No. 62/286,016, filed on Jan. 22, 2016.

(51) Int. Cl. C12M 1/26; C12N 13/00; C12M 3/06; C12M 1/00

(52) U.S. Cl. CPC C12M 33/08; C12N 13/00; C12M 47/04; C12M 23/20; C12M 23/16

(57) ABSTRACT

Methods and devices for manipulating one or more particles (e.g., cells) in three dimensions using surface acoustic waves is described. Methods and devices for printing or more biological cells onto a substrate using surface acoustic waves are also provided.

### RELATED APPLICATIONS

The present application claims priority under 35 U.S.C. § 119(e) to U.S. Provisional Application U.S. Ser. No. 62/286,016, filed on Jan. 22, 2016, which is incorporated herein by reference.

### FEDERALLY SPONSORED RESEARCH

This invention was made with government support under grants 1R33EB019785-01, 1 R01 GM112048-01A1 and U01HL114476 awarded by the National Institutes of Health and government support under grant DMR-0820404 awarded by National Science Foundation. The government has certain rights in the invention.

### BACKGROUND OF THE INVENTION

The ability to precisely manipulate living cells in three-dimensions, one cell at a time, offers many possible applications in regenerative medicine, tissue engineering, neuroscience, and biophysics (1-3). However, current bio-printing methods are generally hampered by the need to reconstruct and mimic three-dimensional (3D) cell-to-cell communications and cell-environment interactions. Due to this constraint, bio-printing requires accurate reproduction of multicellular architecture (4, 5). Several approaches have been developed to produce complex cell patterns, clusters, assembled arrays, and even tissue structures. These approaches use many disparate technologies which include: optics, magnetic and electrical fields, injection printing, physical or geometric constraints, or surface engineering (6-11). However, there is currently a paucity of a single method that can facilitate the formation of complex multicellular structures with high precision, high versatility, multiple dimensionality, and single cell resolution, while maintaining cell viability, integrity, and function. As a result, there is a critical need to develop new methods that seek to overcome these limitations.

### SUMMARY OF THE INVENTION

Aspects of the disclosure relate to methods and devices for manipulating one or more particles (e.g., cells) in three dimensions using surface acoustic waves. Aspects of the disclosure are based, at least in part, on the surprising discovery that a two-dimensional array of acoustic wave generators surrounding a microfluidic reservoir was capable of manipulating particles in the reservoir along three mutually orthogonal axes. In some embodiments, the methods provided herein utilize standing-wave phase shifts (e.g., from acoustic wave generators) to move particles or cells in the same plane as the acoustic wave generators, while the amplitude of acoustic vibrations is used to control particle motion along an orthogonal plane to the acoustic wave generators. It is demonstrated, through controlled experiments guided by simulations, how acoustic vibrations result in micro-manipulations in a microfluidic chamber by invoking physical principles that underlie the formation and regulation of complex, volumetric trapping nodes of particles and biological cells. The devices and methods provided herein can be used to pick up, translate, and print single cells and cell assemblies to create 2D and 3D structures in a precise, noninvasive, label-free, and contact-free manner.

The present disclosure, in one aspect, includes a method of manipulating one or more particles in a reservoir in three dimensions, wherein the reservoir is disposed on a substrate, the method comprising: generating a first surface acoustic wave (SAW) and a second SAW along a first axis of the substrate, wherein the first SAW and the second SAW are generated from opposite sides of the reservoir; generating a third SAW and a fourth SAW along a second axis of the substrate, wherein the third SAW and the fourth SAW are generated from opposite sides of the reservoir, and wherein the first axis and the second axis intersect in the reservoir; manipulating the one or more particles in the reservoir to move along the first axis and/or the second axis by varying a frequency and/or a phase of at least one of the first, the second, the third, and/or the fourth SAW; and manipulating the one or more particles in the reservoir to move along an axis that is orthogonal to the first axis and the second axis by varying an acoustic power of at least one of the first, the second, the third, and/or the fourth SAW. In some embodiments, the first axis is at an oblique angle relative to the second axis.

In another embodiment, the first axis is at a non-oblique angle relative to the second axis. In yet another embodiment, the first axis is at an angle ranging from 1-90 degrees relative to the second axis. In some embodiments, the first axis is at an angle ranging from 45-90 degrees relative to the second axis. In another embodiment, the first axis is at an angle ranging from 75-90 degrees relative to the second axis.

In some embodiments, the first acoustic wave, the second acoustic wave, the third acoustic wave, and the fourth acoustic wave are generated by a first, a second, a third, and a fourth, interdigital transducer (IDT). In another embodiment, the first acoustic wave, the second acoustic wave, the third acoustic wave, and the fourth acoustic wave are generated by a first, a second, a third, and a fourth segmented interdigital transducer (S-IDT).

In some embodiments, the reservoir comprises a fluid. In another embodiment, the reservoir comprises a cell culture medium.

In some embodiments, the one or more particles comprise one or more organic particles, inorganic particles, biological cells, or microorganisms.

In some embodiments, the substrate is a piezoelectric substrate. In other embodiments, the substrate comprises LiNbO₃.

In some embodiments, the reservoir comprises at least one inlet. In other embodiments, the reservoir comprises at least one outlet. In another embodiment, the reservoir comprises at least one inlet and at least one outlet.

In some embodiments, the frequency of any one of the first, the second, the third, and/or the fourth surface acoustic waves is increased. In other embodiments, the frequency of any one of the first, the second, the third, and/or the fourth surface acoustic waves is increased by at least 0.001 MHz, 0.01 MHz, 0.1 MHz, 0.5 MHz, 1 MHz, 2 MHz, 5 MHz or 10 MHz. In another embodiment, the frequency of any one of the first, the second, the third, and/or the fourth surface acoustic waves is decreased. In some embodiments, the frequency of any one of the first, the second, the third, and/or the fourth surface acoustic waves is decreased by at least 0.001 MHz, 0.01 MHz, 0.1 MHz, 0.5 MHz, 1 MHz, 2 MHz, 5 MHz or 10 MHz.

In some embodiments, the phase of any one of the first, the second, the third, and/or the fourth surface acoustic waves is increased. In another embodiment, the phase of any one of the first, the second, the third, and/or the fourth surface acoustic waves is increased by at least 0.1 degree, 1 degree, 2 degrees, 5 degrees, 10 degrees, 15 degrees, or 20 degrees. In other embodiments, the phase of any one of the first, the second, the third, and/or the fourth surface acoustic waves is decreased. In some embodiments, the phase of any one of the first, the second, the third, and/or the fourth surface acoustic waves is decreased by at least 0.1 degree, 1 degree, 2 degrees, 5 degrees, 10 degrees, 15 degrees, or 20 degrees.

In some embodiments, the acoustic power of any one of the first, the second, the third, and/or the fourth surface acoustic waves is increased. In other embodiments, the acoustic power of any one of the first, the second, the third, and/or the fourth surface acoustic waves is increased by at least 0.1 mW, 1 mW, 10 mW, 100 mW, 1000 mW, or 2000 mW. In another embodiment, the acoustic power of any one of the first, the second, the third, and/or the fourth surface acoustic waves is decreased. In some embodiments, the acoustic power of any one of the first, the second, the third, and/or the fourth surface acoustic waves is decreased by at least 0.1 mW, 1 mW, 10 mW, 100 mW, 1000 mW, or 2000 mW.

In some embodiments, the one or more particles in the reservoir are rotated. In another embodiment, the one or more particles in the reservoir are rotated by controlling the direction and/or the velocity of acoustic streaming by adjusting the vibrations.

In some embodiments, imaging one or more of the one or more particles. In another embodiment, the one or more particles are imaged in 3D.

In some aspects, the present disclosure includes a device for manipulating one or more particles in three dimensions, comprising: a reservoir on a substrate; a first pair of surface acoustic wave (SAW) generators, having a first variable power/frequency SAW generator that vibrates the substrate in response to a first input signal and a second variable power/frequency SAW generator that vibrates the substrate in response to a second input signal, wherein the first and second variable power/frequency SAW generators are disposed on the substrate and on opposing sides of the reservoir to generate surface acoustic waves within the reservoir having a first SAW path; a second pair of surface acoustic wave (SAW) generators, having a third variable power/frequency SAW generator that vibrates the substrate in response to a third input signal and a fourth variable power/frequency SAW generator that vibrates the substrate in response to a fourth input signal, wherein the third and fourth variable power/frequency SAW generators are disposed on the substrate and on opposing sides of the reservoir to generate surface acoustic waves within the reservoir having a second SAW path; wherein the first SAW path and the second SAW path are different; and wherein the first, second, third and fourth input signals have an input power, a frequency and a phase.

In some embodiments, the device further comprises an electronic control circuit providing the first, second, third, and fourth input signals to the first, second, third, and fourth variable power/frequency SAW generators, wherein the electronic control circuit is configured to independently vary the input power, the frequency, and the phase of each of the first, second, third and fourth input signals.

In some embodiments, the first pair of surface acoustic wave (SAW) generators is connected to a first radio-frequency (RF) signal generator and the second pair of surface acoustic wave (SAW) generators are connected to a second radio-frequency (RF) signal generator. In other embodiments, the first and second radio-frequency (RF) signal generators each comprise two amplifiers.

In some embodiments, the electronic control circuit is configured to vary the input power from 0.1 mW to 2000 mW. In other embodiments, the electronic control circuit is configured to vary the input power from 1 mW to 1500 mW. In another embodiment, the electronic control circuit is configured to vary the frequency from 1 MHz to 1000 MHz. In other embodiments, the electronic control circuit is configured to vary the frequency from 5 MHz to 50 MHz. In some embodiments, the electronic control circuit is configured to vary the phase by at least 1 degree.

In some embodiments, the reservoir comprises a second substrate. In other embodiments, the second substrate is a scaffold. In another embodiment, the second scaffold is an artificial scaffold. In other embodiments, the second scaffold is a biological scaffold. In another embodiment, the second scaffold comprises one or more biological cells.

In some embodiments, the reservoir comprises a fluid. In other embodiments, the reservoir comprises a cell culture medium.

In some embodiments, the one or more particles comprise one or more organic particles, inorganic particles, biological cells, or microorganisms.

In some embodiments, the SAW generator is an interdigital transducer (IDT). In other embodiments, the SAW generator is a segmented interdigital transducer (S-IDT).

In some embodiments, the substrate is a piezoelectric substrate. In other embodiments, the substrate comprises LiNbO₃. In another embodiment, the substrate comprises a wall of the reservoir.

In some embodiments, one or more of the first, the second, the third, or the fourth variable power/frequency SAW generators are configured to emit an acoustic output ranging from 0.1-40 dBm.

In some embodiments, the reservoir comprises at least one inlet. In another embodiment, the reservoir comprises at least one outlet. In other embodiments, the reservoir comprises at least one inlet and at least one outlet.

In some embodiments, the first SAW path is disposed at an oblique angle relative to the second SAW path. In other embodiments, the first SAW path is disposed at a non-oblique angle relative to the second SAW path. In another embodiment, the first SAW path is disposed at an angle ranging from 1-90 degrees relative to the second SAW path. In other embodiments, the first SAW path is disposed at an angle ranging from 45-90 degrees relative to the second SAW path. In another embodiment, the first SAW path is disposed at an angle ranging from 75-90 degrees relative to the second SAW path.

In some embodiments, the reservoir has a depth ranging from 1 μm to 100 mm. In other embodiments, the reservoir has a length and/or width ranging from 1 μm to 100 mm. In another embodiment, the reservoir has a volume ranging from 0.1 mm³ to 50000 mm³.

The present disclosure, in some aspects, includes a method of manipulating one or more particles in three dimensions using tunable surface acoustic waves, the method comprising: introducing a fluid suspension comprising one or more particles to the reservoir of any one of the device described herein, wherein the first SAW path denotes a first axis, the second SAW path denotes a second axis and a path orthogonal to the first and second SAW paths denotes a third axis; generating surface acoustic waves in the fluid suspension from the first and second pair of SAW generators; manipulating the one or more particles to move along the first axis and/or the second axis by adjusting the frequency and/or phase of one or more of the first, second, third and/or fourth input signals; and manipulating the one or more particles to move along the third axis by adjusting the input power of one or more of the first, second, third and/or fourth input signals.

In some embodiments, the frequency of any one of the first, the second, the third, and/or the fourth input signals is increased. In other embodiments, the frequency of any one of the first, the second, the third, and/or the fourth input signals is increased by at least 0.001 MHz, 0.01 MHz, 0.1 MHz, 0.5 MHz, 1 MHz, 2 MHz, 5 MHz or 10 MHz. In another embodiment, the frequency of any one of the first, the second, the third, and/or the fourth input signals is decreased. In other embodiments, the frequency of any one of the first, the second, the third, and/or the fourth input signals is decreased by at least 0.001 MHz, 0.01 MHz, 0.1 MHz, 0.5 MHz, 1 MHz, 2 MHz, 5 MHz or 10 MHz.

In some embodiments, the phase of any one of the first, the second, the third, and/or the fourth input signals is increased. In other embodiments, the phase of any one of the first, the second, the third, and/or the fourth input signals is increased by at least 0.1 degree, 1 degree, 2 degrees, 5 degrees, 10 degrees, 15 degrees, or 20 degrees. In another embodiment, the phase of any one of the first, the second, the third, and/or the fourth input signals is decreased. In other embodiments, the phase of any one of the first, the second, the third, and/or the fourth input signals is decreased by at least 0.1 degree, 1 degree, 2 degrees, 5 degrees, 10 degrees, 15 degrees, or 20 degrees.

In some embodiments, the acoustic power of any one of the first, the second, the third, and/or the fourth input signals is increased. In other embodiments, the acoustic power of any one of the first, the second, the third, and/or the fourth input signals is increased by at least 0.1 mW, 1 mW, 10 mW, 100 mW, 1000 mW, or 2000 mW. In another embodiment, the acoustic power of any one of the first, the second, the third, and/or the fourth input signals is decreased. In other embodiments, the acoustic power of any one of the first, the second, the third, and/or the fourth input signals is decreased by at least 0.1 mW, 1 mW, 10 mW, 100 mW, 1000 mW, or 2000 mW.

In some embodiments, the fluid suspension comprises a cell culture medium.

In some embodiments, the one or more particles comprise one or more organic particles, inorganic particles, biological cells, or microorganisms.

Additional aspects of the present disclosure include a method of printing one or more biological cells onto a substrate, the method comprising: providing a reservoir disposed on a substrate; generating a first surface acoustic wave (SAW) and a second SAW along a first axis of the substrate, wherein the first SAW and the second SAW are generated from opposite sides of the reservoir; generating a third SAW and a fourth SAW along a second axis of the substrate, wherein the third SAW and the fourth SAW are generated from opposite sides of the reservoir, and wherein the first axis and the second axis intersect in the reservoir; manipulating the one or more biological cells in the reservoir to move along the first axis and/or the second axis by varying a frequency and/or a phase of at least one of the first, the second, the third, and/or the fourth SAW; and manipulating the one or more biological cells in the reservoir to move along an axis that is orthogonal to the first axis and the second axis by varying an acoustic power of at least one of the first, the second, the third, and/or the fourth SAW; and depositing the one or more cells onto a cell substrate.

In some embodiments, the cell substrate is the substrate that the reservoir is disposed on. In other embodiments, the reservoir comprises a second substrate. In another embodiment, the second substrate is a scaffold. In other embodiments, the second substrate is an artificial scaffold. In another embodiment, the second substrate is a biological scaffold. In other embodiments, the cell substrate or the second substrate comprises one or more biological cells. In another embodiment, the cell substrate or the second substrate comprises collagen, fibronectin, an RGD peptide, an extracellular matrix (ECM) protein, or a growth factor.

In some embodiments, one or more cells are deposited onto the cell substrate or the second substrate within 1 uM of a target site on the cell substrate or the second substrate. In another embodiment, one or more cells are deposited on top of a second cell, wherein the second cell is in contact with the cell substrate or the second substrate.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A to 1B show a non-limiting illustration of an embodiment of a three-dimensional (3D) acoustic tweezers device and a 3D trapping node: (FIG. 1A) shows a non-limiting configuration of planar surface acoustic wave generators, used to generate volumetric nodes, surrounding a microfluidic experimental area. The insert image shows a single particle within a “3D trapping node” which can be independently manipulated along the X, Y, or Z axis. (FIG. 1B) shows numerical simulation results mapping an acoustic field around a particle that shows the physical operating principle for the 3D acoustic tweezers. The exemplary 3D trapping node in the microfluidic chamber is created by two superimposed, orthogonal, standing surface acoustic waves and the induced acoustic streaming.

FIGS. 2A to 2D show studies of the acoustic radiation force: (FIG. 2A) Is an exemplary illustration showing how a standing surface acoustic wave enables particle manipulation in a microfluidic chamber. The longitudinal and transverse vibrations are generated by the propagation of standing waves along the X axis on a 128° YX lithium niobate substrate which enables displacement motions along the surface. These displacements introduce an acoustic field and acoustic streaming in the microfluidic chamber which can push and levitate suspended objects. Acoustic radiation force (ARF) is indicated by dotted arrows. Acoustic streaming (AS) is indicated by curved arrows. (FIG. 2B) Are exemplary numerical simulation results showing the distribution of Gor'kov potential and the acoustic radiation force along the X-Z plane induced by the transverse vibrations. The shaded regions indicating a high Gor'kov potential are labeled “High Gor'kov”, while the shaded regions in between indicate a low Gor'kov potential. The arrows from the high Gor'kov regions to the low Gor'kov regions show the direction of the acoustic radiation forces. (FIG. 2C) Is an exemplary diagram of the radiation force distribution between the periodic array of nodes and antinodes, and the correlated experimental manipulation of 10.1 μm polystyrene particles along the X-Y plane without, and then with, an applied standing surface acoustic wave. (FIG. 2D) Are experimental results showing the dependence of the acoustic radiation force on the input power. Scale bar: 100 μm.

FIGS. 3A to 3D show an exemplary study of acoustic streaming: (FIG. 3A) Shows simulation results of the acoustic streaming patterns in the X-Z plane, as induced by both the longitudinal and transverse vibrations. The streaming lines rotate clockwise or counterclockwise from a displacement node to two neighboring displacement antinodes. The arrows indicate the rotation directions. The shading labeled with an “*” nearby the substrate surface indicate the high magnitudes of the streaming velocities, whereas the remaining shading above the high magnitude areas shows low velocities. (FIG. 3B) Shows simulation results of the distribution of Gor'kov potential overlaid on top of the acoustic streaming results in a microfluidic chamber along the X-Z plane. The shaded regions indicating a high Gor'kov potential are labeled, while the shaded regions in between indicate a low Gor'kov potential. (FIG. 3C) Shows a visualization of the acoustic streaming pattern along a periodic array of displacement nodes and antinodes, and the overlapped experimental acoustic streaming lines along the X-Y plane, under a standing surface acoustic wave. Path lines formed from 1 μm red fluorescent polystyrene particles as they flow up from the pre-marked displacement nodes and rotating as the simulations predicted. (FIG. 3D) Are experimental results of the dependence of the magnitudes of streaming velocities on input power. Scale bar: 100 μm.

FIGS. 4A to 4D show vertical (Z-axis) acoustic manipulation: (FIG. 4A) Shows the calculated trapping node induced by a standing acoustic wave in the XY plane within a microfluidic chamber, and an illustration of the forces along the Z direction in the trapping node. (FIG. 4B) Shows theoretical calculations and (FIG. 4C) an experimental demonstration of vertical position control of a single 10.1 μm polystyrene particle by varying the input power. The images taken in the XY plane show the stable trapped single particle was lifted up and through the microscope's focal point by increasing the input power from 200 mW to 1500 mW. (FIG. 4D) Are experimental results showing the vertical positions of particles with different sizes (4.2 μm, 7.3 μm, 10.1 μm) under different input powers, which are consistent with the predictions from the numerical model. Scale bar: 20 μm.

FIGS. 5A to 5C show 3D acoustic manipulation: (FIG. 5A) Is an illustration of the 2D distribution of transverse vibrations on the substrate. Dotted lines represent displacement nodes of transverse vibrations that have minimal vibration amplitudes (the solid lines represent displacement antinodes). (FIG. 5B) Shows superimposed images of 3D trapping and streaming patterns. 10 μm green fluorescent polystyrene particles, labeled by “*”, were trapped into a suspended array of single dots, and 1 μm red fluorescent polystyrene particles tracked the motion of the acoustic streaming vortex (the arrows indicate the direction of the streaming near the substrate surface). (FIG. 5C) Is an image sequence showing the 3D trapping and manipulation of particles with a stable array configuration. Exemplary trapping nodes (indicated by dotted circles) can levitate particles along the Z direction (moving into focus) and pushing particles together, transporting them along Y axis, and dropping them back towards the substrate (moving out of focus). Scale bar: 100 μm.

FIGS. 6A to 6B show printing of living cells with 3D acoustic tweezers: (FIG. 6A) Shows single cell printing. After previously deposited 3T3 cells were attached to the substrate, another single cell was picked up, transported and dropped at a desired location on the substrate, as indicated by the black arrow (or on top of another cell, as indicated with the white arrow). The single cell adhered and spread along the surface. (FIG. 6B) Shows the formation of arbitrary cell culture patterns forming a “3” “D” “A” and “T” by printing of single HeLa S3 cells via 3D acoustic tweezers. Scale bar: 20 μm.

FIGS. 7A to 7B show an exemplary scheme of 1D SSAW device and 2D fluid domain for modelling. (FIG. 7A) Shows a cross section of the 1D SSAW device, comprising of a 128° YX LiNO₃ substrate with one pair of IDTs on its surface and a PDMS chamber filled with fluid. (FIG. 7B) Shows a 2D fluidic domain used for numerical modelling. Two decaying LSAWs propagate in opposite directions to form the actuation boundary at the bottom. The other boundaries are lossy-wall boundaries. The dimensions of this 2D modelled domain are 1800 μm×100 μm (Width×Height).

FIGS. 8A to 8D show an exemplary calibration of vibration amplitude. (FIG. 8A) Is a schematic of an exemplary 1D SSAW microfluidic device. Two traveling waves are propagating towards each other and superposed to form a standing wave along the X axis of a 128° YX cut lithium niobate substrate. Before entering into the microfluidic chamber, each wave propagates through a PDMS chamber wall which results in a decay of the vibration amplitude. (FIG. 8B) Is an exemplary curve showing the calibrated dependence of the amplitude of the LSAW (in the fluidic chamber) on the input power. (FIG. 8C) Is an illustration of an exemplary setup for directly measuring the amplitude of the longitudinal vibration of the SSAW. An additional 4 mm×4 mm gold film with a thickness of 200 nm is deposited in the middle of the pair of IDTs to reflect light during the optical measurement. (FIG. 8D) Shows a results graph of the amplitude of the longitudinal vibrations under different input powers by directly measuring the SAW vibrations on the substrate with an optical vibrometer system.

FIG. 9 shows viability of cells after 3D acoustic printing. Image sequences show that the HeLa S3 cells are dividing and proliferating after being printed into the cell culture pattern of the letter “A”. Scale bar: 50 μm.

FIG. 10 is a schematic of an exemplary device for manipulating one or more particles in three dimensions.

FIG. 11 is a schematic of an exemplary pair of segmented interdigital transducers (S-IDTs) on opposite sides of a reservoir.

### DETAILED DESCRIPTION OF THE INVENTION

The ability of surface acoustic waves to trap and manipulate micrometer-scale particles and biological cells has led to many applications involving “acoustic tweezers” in biology, chemistry, engineering and medicine. Provided herein are three-dimensional (3D) acoustic tweezers which use surface acoustic waves to create 3D trapping nodes for the capture and manipulation of particles (e.g., microparticles and cells) along three mutually orthogonal axes. The methods provided herein utilize standing-wave phase shifts to move particles or cells in-plane, while the amplitude of acoustic vibrations is used to control particle motion along an orthogonal plane. It is demonstrated, through controlled experiments guided by simulations, how acoustic vibrations result in micro-manipulations in a microfluidic chamber by invoking physical principles that underlie the formation and regulation of complex, volumetric trapping nodes of particles and biological cells. It is further shown how 3D acoustic tweezers can be employed to pick up, translate, and print single cells and cell assemblies to create 2D and 3D structures in a precise, noninvasive, label-free, and contact-free manner.

### Methods for Manipulating One or More Particles in 3D

Aspects of the disclosure relate to methods of manipulating one or more particles in a reservoir in 3 dimensions (3D) using surface acoustic waves. As used herein, the term “manipulating” refers to moving one or more of the particles in a reservoir. In some embodiments, one or more particles are moved along 3 mutually orthogonal axes within the reservoir. The one or more particles may be rotated and/or moved in one or more directions. In some embodiments, one or more particles are moved along a first axis, for example, along an axis between two opposing surface acoustic waves (e.g., a first SAW and a second SAW). In some embodiments, one or more particles are moved along a second axis that is in the same plane as the first axis, but where the second axis intersects the first axis in the reservoir. In some embodiments, the second axis is between a separate pair of opposing surface acoustic waves (e.g., a third SAW and a fourth SAW). In some embodiments, the surface acoustic waves are generated in a pattern such that a first axis (e.g., between a first and a second opposing SAW) and a second axis (e.g., between a third and a fourth SAW) intersect in the reservoir. In some embodiments, the first axis is at a right angle relative to the second axis. In some embodiments, the first axis is at an oblique angle relative to the second axis In some embodiments, the first axis is from 1 degree to 5 degrees, from 1 degree to 10 degrees, from 1 degree to 20 degrees, from 1 degree to 40 degrees, from 1 degree to 60 degrees, from 1 degree to 80 degrees, from 5 degrees to 10 degrees, from 5 degrees to 20 degrees, from 5 degrees to 40 degrees, from 5 degrees to 60 degrees, from 5 degrees to 80 degrees, from 5 degrees to 90 degrees, from 10 degrees to 20 degrees, from 10 degrees to 40 degrees, from 10 degrees to 60 degrees, from 10 degrees to 80 degrees, from 10 degrees to 90 degrees, from 20 degrees to 40 degrees, from 20 degrees to 60 degrees, from 20 degrees to 80 degrees, from 20 degrees to 90 degrees, from 40 degrees to 60 degrees, from 40 degrees to 80 degrees, from 40 degrees to 90 degrees, from 60 degrees to 80 degrees, from 60 degrees to 90 degrees, or from 80 degrees to 90 degrees relative to the second axis.

In some embodiments, one or more particles are moved along the first axis and/or the second axis by varying (e.g., increasing or decreasing) a frequency and/or a phase of one or more of the first, second, third, and/or fourth SAW. In some embodiments, one or more particles is moved along the first axis by increasing or decreasing the frequency of the first SAW. In some embodiments, one or more particles is moved along the first axis by increasing or decreasing the frequency of the second SAW. In some embodiments, one or more particles is moved along the second axis by increasing or decreasing the frequency of the third SAW. In some embodiments, one or more particles is moved along the second axis by increasing or decreasing the frequency of the fourth SAW. In some embodiments, one or more particles is moved along the first axis by increasing or decreasing the phase of the first SAW. In some embodiments, one or more particles is moved along the first axis by increasing or decreasing the phase of the second SAW. In some embodiments, one or more particles is moved along the second axis by increasing or decreasing the phase of the third SAW. In some embodiments, one or more particles is moved along the second axis by increasing or decreasing the frequency of the fourth SAW. It should be appreciated that the one or more particles may move along any of the 3 mutually orthogonal axes simultaneously. In some embodiments, one or more particles are moved along the first axis and the second axis simultaneously, for example, by varying the frequency and/or phase the first SAW at the same time as the third SAW or the fourth SAW. As another example, one or more particles are moved along the first axis and the second axis simultaneously by varying the frequency and/or phase of the second SAW at the same time as the third SAW or the fourth SAW.

In some embodiments, one or more particles are moved along an axis that is orthogonal to the first axis and the second axis by varying (e.g., increasing or decreasing) an acoustic power of one or more of the first, second, third, and/or fourth SAW. In some embodiments, an acoustic power of the first SAW is increased or decreased. In some embodiments, an acoustic power of the second SAW is increased or decreased. In some embodiments, an acoustic power of the third SAW is increased or decreased. In some embodiments, an acoustic power of the fourth SAW is increased or decreased. In some embodiments, an acoustic power of the first SAW and the second SAW is increased or decreased. In some embodiments, an acoustic power of the first SAW and the third SAW is increased or decreased. In some embodiments, an acoustic power of the first SAW and the fourth SAW is increased or decreased. In some embodiments, an acoustic power of the second SAW and the third SAW is increased or decreased. In some embodiments, an acoustic power of the second SAW and the fourth SAW is increased or decreased. In some embodiments, an acoustic power of the third SAW and the fourth SAW is increased or decreased. In some embodiments, an acoustic power of the first SAW the second SAW and the third SAW is increased or decreased. In some embodiments, an acoustic power of the first SAW the second SAW and the fourth SAW is increased or decreased. In some embodiments, an acoustic power of the first SAW the third SAW and the fourth SAW is increased or decreased. In some embodiments, an acoustic power of the second SAW the third SAW and the fourth SAW is increased or decreased. In some embodiments, an acoustic power of the first SAW the second SAW the third SAW and the fourth SAW is increased or decreased.

In some embodiments, the methods comprise manipulating one or more particles in a reservoir. As used herein, the term “particle” refers to a quantity of matter having one or more physical properties such as volume or mass. In some embodiments, the particle is a synthetic object such as, for example, a polymeric bead (e.g., a polyethylene bead or a polystyrene bead), a micelle, a liposome, etc. A particle may be a microparticle or nanoparticle. The term “microparticle,” as used herein, refers to a particle having an average diameter on the order of micrometers (between about 1 micrometer and about 1 mm), while the term “nanoparticle” refers to a particle having an average diameter on the order of nanometers (between about 1 nm and about 1 micrometer). The particle may also have any shape or size. For instance, the particle may have an average diameter of less than about 5 mm, less than about 2 mm, less than about 1 mm, or less than about 500 μm, less than about 200 μm, less than about 100 μm, less than about 60 μm, less than about 50 μm, less than about 40 μm, less than about 30 μm, less than about 25 μm, less than about 10 μm, less than about 3 μm, less than about 1 μm, less than about 300 nm, less than about 100 nm, less than about 30 nm, or less than about 10 nm. In some embodiments, the particle has an average diameter from 1 nm to 5 mm. The particles may be spherical or non-spherical. The average diameter of a non-spherical particle is the diameter of a perfect sphere having the same volume as the non-spherical particle.

A particle may be a biological object such as, for example, a vesicle, a eukaryotic cell, a prokaryotic cell, an organelle, a cell fragment (e.g., a platelet), a virus, a biomolecular aggregate, or an organism (e.g., a C. elegans organism). Eukaryotic cells may be primary cells isolated from any tissue or organ (e.g., connective, nervous, muscle, fat or epithelial tissue). The cells may be mesenchymal, ectodermal, or endodermal. The cells may be nucleated or non-nucleated.

In some embodiments, the particle is a cell, e.g., a red blood cell, a white blood cell, a stem cell, a cancer cell, an epithelial cell (e.g., epithelial cells of the cervix, pancreas, breast or bladder), a B cell, a T cell, or a plasma cell. Cells may be derived from, or contained in, isolated connective, nervous, muscle, fat or epithelial tissue. The connective tissue may be, for example, blood, bone, ligament, cartilage, tendon, or adipose tissue. The muscle tissue may be vascular smooth muscle, heart smooth muscle, or skeletal muscle, for example. The epithelial tissue may be of the blood vessels, ducts of submandibular glands, attached gingiva, dorsum of tongue, hard palate, esophagus, pancreas, adrenal glands, pituitary glands, prostate, liver, thyroid, stomach, small intestine, large intestine, rectum, anus, gallbladder, thyroid follicles, ependyma, lymph vessel, skin, sweat gland ducts, mesothelium of body cavities, ovaries, fallopian tubes, uterus, endometrium, cervix (endocervix), cervix (ectocervix), vagina, labia majora, tubuli recti, rete testis, ductuli efferentes, epididymis, vas deferens, ejaculatory duct, bulbourethral glands, seminal vesicle, oropharynx, larynx, vocal cords, trachea, respiratory bronchioles, cornea, nose, proximal convoluted tubule of kidney, ascending thin limb of kidney, distal convoluted tubule of kidney, collecting duct of kidney, renal pelvis, ureter, urinary bladder, prostatic urethra, membranous urethra, penile urethra, or external urethral orifice, for example.

In some embodiments, cells may be from an established cell line. For example, in some embodiments, cells may be HeLA, NCI60, DU145, Lncap, MCF-7, MDA-MB-438, PC3, T47D, THP-1, U87, SHSY5Y, Saos-2, KBM-7, Vero, GH3, PC12, MC3T3, 3T3, 293T, HEK-293 or MDCK cells. However, it should be appreciated that any cell line may be used.

The cells may be any mammalian cells. The cells may be any human cells. The cells may be selected from the group consisting of lymphocytes, B cells, T cells, cytotoxic T cells, natural killer T cells, regulatory T cells, T helper cells, myeloid cells, granulocytes, basophil granulocytes, eosinophil granulocytes, neutrophil granulocytes, hypersegmented neutrophils, monocytes, macrophages, reticulocytes, platelets, mast cells, thrombocytes, megakaryocytes, dendritic cells, thyroid cells, thyroid epithelial cells, parafollicular cells, parathyroid cells, parathyroid chief cells, oxyphil cells, adrenal cells, chromaffin cells, pineal cells, pinealocytes, glial cells, glioblasts, astrocytes, oligodendrocytes, microglial cells, magnocellular neurosecretory cells, stellate cells, boettcher cells; pituitary cells, gonadotropes, corticotropes, thyrotropes, somatotrope, lactotrophs, pneumocyte, type I pneumocytes, type II pneumocytes, Clara cells; goblet cells, alveolar macrophages, myocardiocytes, pericytes, gastric cells, gastric chief cells, parietal cells, goblet cells, paneth cells, G cells, D cells, ECL cells, I cells, K cells, S cells, enteroendocrine cells, enterochromaffin cells, APUD cell, liver cells, hepatocytes, Kupffer cells, bone cells, osteoblasts, osteocytes, osteoclast, odontoblasts, cementoblasts, ameloblasts, cartilage cells, chondroblasts, chondrocytes, skin cells, hair cells, trichocytes, keratinocytes, melanocytes, nevus cells, muscle cells, myocytes, myoblasts, myotubes, adipocyte, fibroblasts, tendon cells, podocytes, juxtaglomerular cells, intraglomerular mesangial cells, extraglomerular mesangial cells, kidney cells, kidney cells, macula densa cells, spermatozoa, sertoli cells, leydig cells, oocytes, and mixtures thereof.

The cells may also be isolated from a healthy tissue or a diseased tissue, e.g., a cancer.

Accordingly, the cells may be cancer cells. For example, the cells may be isolated or derived from any of the following types of cancers: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia, e.g., B Cell CLL; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Cancer cells may be cells derived from any stage of cancer progression including, for example, precancerous cells, cancerous cells, and metastatic cells. Cancer cells also include cells from a primary tumor, secondary tumor or metastasis.

The cells may be selected from the group consisting of cord-blood cells, stem cells, embryonic stem cells, adult stem cells, cancer stem cells, progenitor cells, autologous cells, isograft cells, allograft cells, xenograft cells, and genetically engineered cells. The cells may be induced progenitor cells. The cells may be cells isolated from a subject, e.g., a donor subject, which have been transfected with a stem cell associated gene to induce pluripotency in the cells. The stem cell-associated genes may be selected from the group consisting of Oct3, Oct4, Sox1, Sox2, Sox3, Sox15, Klf1, Klf2, Klf4, Klf5, Nanog, Lin28, C-Myc, L-Myc, and N-Myc. The cells may be cells which have been isolated from a subject, transfected with a stem cell associated gene to induce pluripotency, and differentiated along a predetermined cell lineage.

In some embodiments, the cells are prokaryotic cells. Prokaryotic cells may be from any phyla, including Aquificae, Bacteroids, Chlorobia, Chrysogenetes, Cyanobacteria, Fibrobacter, Firmicutes, Flavobacteria, Fusobacteria, Proteobacteria, Sphingobacteria, Spirochaetes, Thermomicrobia, and/or Xenobacteria, among others. Such bacteria may be gram-negative, gram-positive, harmful, beneficial, and/or pathogenic. Exemplary prokaryotic cells may include E. coli, S. typhimurium, B subtilis, S. aureus, C. perfiingens, V. parahaemolyticus, and/or B. anthracis, among others.

In another example, particles are viruses (or cells infected therewith) including, for example, any DNA, RNA, and/or protein containing particle that infects and/or replicates in cells. The term virus encompasses DNA viruses, RNA viruses, retroviruses, virions, viroids, prions, etc. Exemplary viruses may include HIV, RSV, rabies, hepatitis virus, Epstein-Barr virus, rhinoviruses, bacteriophages, and diseases causing prions. In another example, the particles are organelles.

In some embodiments, the reservoir comprises a fluid. In some embodiments, the fluid is suitable for use in accordance with the methods provided herein. In some embodiments, the fluid comprises water. In some embodiments, the fluid comprises a buffer. In some embodiments, the buffer is glycine-HCl buffer, a glycine-NaOH buffer, a sodium acetate buffer, a buffered saline (e.g., PBS, TBS, TNT, PBT), a cacodylate buffer, a good buffer, a citrate buffer a sorensen's phosphate buffer, a barbital buffer, or a tris buffer. However, additional buffers may be used and are within the scope of this disclosure.

In some embodiments, the reservoir comprises a cell culture medium. In some embodiments, the cell culture medium is suitable for use with any of the cells provided herein. For example, in some embodiments, the cell culture medium may include one or more of MEM, DMEM, IMDM, RPMI1640, 199/109 medium, HamF10/HamF12, or McCoy's 5A. However, additional types of cell culture medium may be used and are within the scope of this disclosure.

In some embodiments, the substrate of the device is a piezoelectric substrate that supports the reservoir and/or one or more surface acoustic wave generators to generate one or more surface acoustic waves. In some embodiments, the piezoelectric substrate is LiNbO₃. However, any suitable piezoelectric substrates may be used and are within the scope of the disclosure. In some embodiments, the LiNbO₃ piezoelectric substrate is a 128° YX-cut LiNbO₃ substrate.

In some embodiments, the reservoir has one or more inlets and/or one or more outlets to allow a fluid (e.g., a fluid comprising one or more particles) to be added to or removed from the reservoir. In some embodiments, the reservoir comprises 1, 2, 3, 4, 5, 6, 7, 8, 9 or 10 inlets. In some embodiments, the reservoir comprises 1, 2, 3, 4, 5, 6, 7, 8, 9 or 10 outlets.

In some embodiments, the methods include increasing and/or decreasing the frequency, phase, and/or acoustic power of one or more of the first, the second, the third, and/or the fourth acoustic waves. In some embodiments, increasing or decreasing the frequency of the first and/or second acoustic waves, which are generated from opposite sides of the reservoir, can be performed to move one or more particles in the reservoir along an axis (e.g., a first axis), denoted by the direction of the first and second acoustic waves. In some embodiments, increasing or decreasing the frequency of the third, and/or fourth acoustic waves, which are generated from opposite sides of the reservoir, can be performed to move one or more particles in the reservoir along another axis (e.g., a second axis), that is in the same plane with the first axis and intersects the first axis. In some embodiments, the second axis is denoted by the direction of the third and fourth acoustic waves. In some embodiments, the first axis and the second axis are orthogonal.

In some embodiments, the frequency of any one of the first, the second, the third, and/or the fourth surface acoustic waves is increased. In some embodiments, the frequency is increased by at least 0.001 MHz, 0.01 MHz, 0.1 MHz, 0.5 MHz, 1 MHz, 2 MHz, 5 MHz or 10 MHz. In some embodiments, the frequency is increased by 0.001 MHz to 0.01 MHz, by 0.001 MHz to 0.1 MHz, by 0.001 MHz to 0.5 MHz, by 0.001 MHz to 1 MHz, by 0.001 MHz to 2 MHz, by 0.001 MHz to 5 MHz, by 0.01 MHz to 0.1 MHz, by 0.01 MHz to 0.5 MHz, by 0.01 MHz to 1 MHz, by 0.01 MHz to 2 MHz, by 0.01 MHz to 5 MHz, by 0.01 MHz to 10 MHz, by 0.1 MHz to 0.5 MHz, by 0.1 MHz to 1 MHz, by 0.1 MHz to 2 MHz, by 0.1 MHz to 5 MHz, by 0.1 MHz to 10 MHz, by 1 MHz to 2 MHz, by 1 MHz to 5 MHz, by 1 MHz to 10 MHz, by 2 MHz to 5 MHz, by 2 MHz to 10 MHz, or by 5 MHz to 10 MHz.

In some embodiments, the frequency of any one of the first, the second, the third, and/or the fourth surface acoustic waves is decreased. In some embodiments, the frequency is decreased by at least 0.001 MHz, 0.01 MHz, 0.1 MHz, 0.5 MHz, 1 MHz, 2 MHz, 5 MHz or 10 MHz. In some embodiments, the frequency is decreased by 0.001 MHz to 0.01 MHz, by 0.001 MHz to 0.1 MHz, by 0.001 MHz to 0.5 MHz, by 0.001 MHz to 1 MHz, by 0.001 MHz to 2 MHz, by 0.001 MHz to 5 MHz, by 0.01 MHz to 0.1 MHz, by 0.01 MHz to 0.5 MHz, by 0.01 MHz to 1 MHz, by 0.01 MHz to 2 MHz, by 0.01 MHz to 5 MHz, by 0.01 MHz to 10 MHz, by 0.1 MHz to 0.5 MHz, by 0.1 MHz to 1 MHz, by 0.1 MHz to 2 MHz, by 0.1 MHz to 5 MHz, by 0.1 MHz to 10 MHz, by 1 MHz to 2 MHz, by 1 MHz to 5 MHz, by 1 MHz to 10 MHz, by 2 MHz to 5 MHz, by 2 MHz to 10 MHz, or by 5 MHz to 10 MHz.

In some embodiments, increasing or decreasing the phase of the first and/or second acoustic waves, which are generated from opposite sides of the reservoir, can be performed to move one or more particles in the reservoir along an axis (e.g., a first axis), denoted by the direction of the first and second acoustic waves. In some embodiments, increasing or decreasing the phase of the third, and/or fourth acoustic waves, which are generated from opposite sides of the reservoir, can be performed to move one or more particles in the reservoir along another axis (e.g., a second axis), that is in the same plane with the first axis and intersects the first axis. In some embodiments, the second axis is denoted by the direction of the third and fourth acoustic waves. In some embodiments, the first axis and the second axis are orthogonal.

In some embodiments, the phase of any one of the first, the second, the third, and/or the fourth surface acoustic waves is increased. In some embodiments, the phase is increased by at least 0.1 degree, 1 degree, 2 degrees, 5 degrees, 10 degrees, 15 degrees, or 20 degrees.

In some embodiments, the phase is decreased by 0.1 degree to 1 degree, by 0.1 degree to 2 degrees, by 0.1 degree to 5 degrees, by 0.1 degree to 10 degrees, by 0.1 degree to 15 degrees, by 1 degree to 2 degrees, by 1 degree to 5 degrees, by 1 degree to 10 degrees, by 1 degree to 15 degrees, by 1 degree to 20 degrees, by 2 degrees to 5 degrees, by 2 degrees to 10 degrees, by 2 degrees to 15 degrees, by 2 degrees to 20 degrees, by 5 degrees to 10 degrees, by 5 degrees to 15 degrees, by 5 degrees to 20 degrees, by 10 degrees to 15 degrees, by 10 degrees to 20 degrees, or by 15 degrees to 20 degrees.

In some embodiments, the phase of any one of the first, the second, the third, and/or the fourth surface acoustic waves is decreased. In some embodiments, the phase is decreased by at least 0.1 degree, 1 degree, 2 degrees, 5 degrees, 10 degrees, 15 degrees, or 20 degrees.

In some embodiments, the phase is decreased by 0.1 degree to 1 degree, by 0.1 degree to 2 degrees, by 0.1 degree to 5 degrees, by 0.1 degree to 10 degrees, by 0.1 degree to 15 degrees, by 1 degree to 2 degrees, by 1 degree to 5 degrees, by 1 degree to 10 degrees, by 1 degree to 15 degrees, by 1 degree to 20 degrees, by 2 degrees to 5 degrees, by 2 degrees to 10 degrees, by 2 degrees to 15 degrees, by 2 degrees to 20 degrees, by 5 degrees to 10 degrees, by 5 degrees to 15 degrees, by 5 degrees to 20 degrees, by 10 degrees to 15 degrees, by 10 degrees to 20 degrees, or by 15 degrees to 20 degrees.

As described herein, changing the frequency and/or phase of the first, second, third and/or fourth surface acoustic waves, which are generated in the same plane, can be performed to move one or more particles in a reservoir along two axes (e.g., in 2D), thus controlling the movement of the one or more particles in the reservoir in two dimensions. In some embodiments, the methods provided herein permit the control of one or more of the particles in the reservoir to move along a third axis that is orthogonal to the first axis and the second axis (e.g., in a direction toward the top, or the bottom of the reservoir). For example, the methods provided herein can be used to move one or more particles along three mutually orthogonal axes. In some embodiments, one or more particles in the reservoir is moved along an axis that is orthogonal to the first axis and the second axis by increasing or decreasing the acoustic power of one or more of the first, the second, the third, and/or the fourth acoustic waves.

In some embodiments, the acoustic power of any one of the first, the second, the third, and/or the fourth surface acoustic waves is increased. In some embodiments, the acoustic power is increased by at least 0.1 mW, 1 mW, 10 mW, 100 mW, 1000 mW, or 2000 mW. In some embodiments, the acoustic power is increased by 0.1 mW to 1 mW, by 0.1 mW to 10 mW, by 0.1 mW to 100 mW, by 0.1 mW to 1000 mW, by 1 mW to 10 mW, by 1 mW to 100 mW, by 1 mW to 1000 mW, by 1 mW to 2000 mW, by 10 mW to 100 mW, by 10 mW to 1000 mW, by 10 mW to 2000 mW, 100 mW to 1000 mW, by 100 mW to 2000 mW, or by 1000 mW to 2000 mW.

In some embodiments, the acoustic power of any one of the first, the second, the third, and/or the fourth surface acoustic waves is decreased. In some embodiments, the acoustic power is decreased by at least 0.1 mW, 1 mW, 10 mW, 100 mW, 1000 mW, or 2000 mW. In some embodiments, the acoustic power is decreased by 0.1 mW to 1 mW, by 0.1 mW to 10 mW, by 0.1 mW to 100 mW, by 0.1 mW to 1000 mW, by 1 mW to 10 mW, by 1 mW to 100 mW, by 1 mW to 1000 mW, by 1 mW to 2000 mW, by 10 mW to 100 mW, by 10 mW to 1000 mW, by 10 mW to 2000 mW, 100 mW to 1000 mW, by 100 mW to 2000 mW, or by 1000 mW to 2000 mW.

In some embodiments, the acoustic power of the first, second, third, and/or fourth surface acoustic waves is increased. In some embodiments, the acoustic power of the first surface acoustic wave is increased. In some embodiments, the acoustic power of the second surface acoustic wave is increased. In some embodiments, the acoustic power of the third surface acoustic wave is increased. In some embodiments, the acoustic power of the fourth surface acoustic wave is increased. In some embodiments, the acoustic power of the first and the second surface acoustic waves are increased. In some embodiments, the acoustic power of the first and the third surface acoustic waves are increased. In some embodiments, the acoustic power of the first and the fourth surface acoustic waves are increased. In some embodiments, the acoustic power of the second and the third surface acoustic waves are increased. In some embodiments, the acoustic power of the second and the fourth surface acoustic waves are increased. In some embodiments, the acoustic power of the third and the fourth surface acoustic waves are increased. In some embodiments, the acoustic power of the first, second and the third surface acoustic waves are increased. In some embodiments, the acoustic power of the first, second and the fourth surface acoustic waves are increased. In some embodiments, the acoustic power of the first, third and the fourth surface acoustic waves are increased. In some embodiments, the acoustic power of the second, third and the fourth surface acoustic waves are increased. In some embodiments, the acoustic power of the first, second, third and the fourth surface acoustic waves are increased.

In some embodiments, the acoustic power of the first, second, third, and/or fourth surface acoustic waves is decreased. In some embodiments, the acoustic power of the first surface acoustic wave is decreased. In some embodiments, the acoustic power of the second surface acoustic wave is decreased. In some embodiments, the acoustic power of the third surface acoustic wave is decreased. In some embodiments, the acoustic power of the fourth surface acoustic wave is decreased. In some embodiments, the acoustic power of the first and the second surface acoustic waves are decreased. In some embodiments, the acoustic power of the first and the third surface acoustic waves are decreased. In some embodiments, the acoustic power of the first and the fourth surface acoustic waves are decreased. In some embodiments, the acoustic power of the second and the third surface acoustic waves are decreased. In some embodiments, the acoustic power of the second and the fourth surface acoustic waves are decreased. In some embodiments, the acoustic power of the third and the fourth surface acoustic waves are decreased. In some embodiments, the acoustic power of the first, second and the third surface acoustic waves are decreased. In some embodiments, the acoustic power of the first, second and the fourth surface acoustic waves are decreased. In some embodiments, the acoustic power of the first, third and the fourth surface acoustic waves are decreased. In some embodiments, the acoustic power of the second, third and the fourth surface acoustic waves are decreased. In some embodiments, the acoustic power of the first, second, third and the fourth surface acoustic waves are decreased.

In some embodiments, the methods provided herein can be performed using any one of the devices provided herein.

Devices for Manipulating One or More particles in 3D

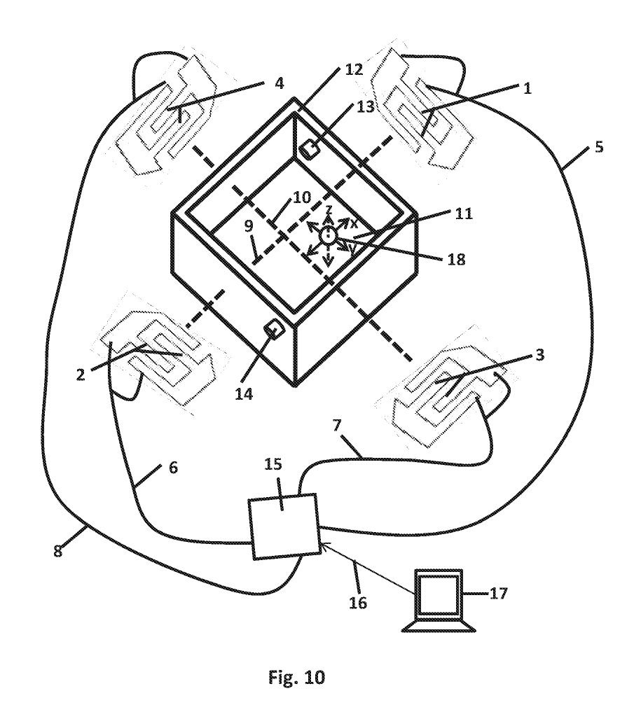

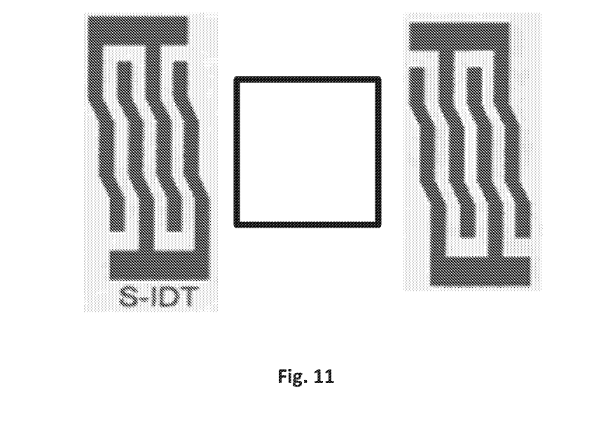

Aspects of the disclosure relate to devices for manipulating/moving one or more particles (e.g., one or more cells) along three orthogonal axes within a reservoir using surface acoustic waves. One non-limiting example of the device is shown in FIG. 10. The embodiment illustrated in FIG. 10 corresponds to a working example fabricated and tested by the inventors, though it represents merely one example of many possible embodiments. The device, in some embodiments, has a reservoir 12 on a substrate 11. A reservoir, having one or more walls, may be bonded to the substrate. In some embodiments, the reservoir is square-shaped, as depicted in FIG. 10. For example, the walls of the reservoir that are bound to the substrate may form a square shape. However, the one or more walls of the reservoir may form any suitable shape. In some embodiments, the walls of the reservoir form a square-shape, a rectangular-shape, a triangular-shape, a polyagonal-shape, a round-shape, or an oval-shape. In some embodiments, the reservoir has a top surface or wall, which may enclose the reservoir.

The reservoir and the substrate may be fabricated using any suitable material or method known in the art. For example, the reservoir may be fabricated using poly-dimethylsiloxane (PDMS) casting protocols, and the substrate may be fabricated from glass or a piezoelectric substrate such as LiNbO₃. However, it should be appreciated that the methods and materials for making the reservoir and substrate provided herein are exemplary and not meant to be limiting. The depth of the reservoir may be any suitable depth for manipulating particles in 3D. In some embodiments, the reservoir has a depth ranging from 1 μm to 100 mm. For example, the reservoir may have a depth ranging from 1 μm to 100 μm, from 1 μm to 500 μm, from 1 μm to 1 mm, from 1 μm to 2.5 mm, from 1 μm to 5 mm, from 1 μm to 10 mm, from 100 μm to 500 μm, from 100 μm to 1 mm, from 100 μm to 2.5 mm, from 100 μm to 5 mm, from 100 μm to 10 mm, from 100 μm to 100 mm, from 500 μm to 1 mm, from 500 μm to 2.5 mm, from 500 μm to 5 mm, from 500 μm to 10 mm, from 500 μm to 100 mm, from 1 mm to 2.5 mm, from 1 mm to 5 mm, from 1 mm to 10 mm, from 1 mm to 100 mm, from 2.5 mm to 5 mm, from 2.5 mm to 10 mm, from 2.5 mm to 100 mm, from 5 mm to 10 mm, from 5 mm to 100 mm, or from 10 mm to 100 mm.

The length or width of the reservoir may be any suitable length or width for manipulating particles in 3D. In some embodiments, the reservoir has a length and/or width ranging from 1 μm to 100 mm. For example, the reservoir may have a depth ranging from 1 μm to 100 μm, from 1 μm to 500 μm, from 1 μm to 1 mm, from 1 μm to 2.5 mm, from 1 μm to 5 mm, from 1 μm to 10 mm, from 100 μm to 500 μm, from 100 μm to 1 mm, from 100 μm to 2.5 mm, from 100 μm to 5 mm, from 100 μm to 10 mm, from 100 μm to 100 mm, from 500 μm to 1 mm, from 500 μm to 2.5 mm, from 500 μm to 5 mm, from 500 μm to 10 mm, from 500 μm to 100 mm, from 1 mm to 2.5 mm, from 1 mm to 5 mm, from 1 mm to 10 mm, from 1 mm to 100 mm, from 2.5 mm to 5 mm, from 2.5 mm to 10 mm, from 2.5 mm to 100 mm, from 5 mm to 10 mm, from 5 mm to 100 mm, or from 10 mm to 100 mm.

The volume of the reservoir may be any suitable volume for manipulating particles in 3D. In some embodiments, the reservoir has a volume ranging from 0.000001 mm³ to 50000 mm³. For example, the reservoir may have a volume ranging from 0.000001 mm³ to 0.00001 mm³, from 0.0001 mm³ to 0.001 mm³, from 0.01 mm³ to 0.1 mm³, from 0.1 mm³ to 1 mm³, 0.1 mm³ to 1 mm³, from 0.1 mm³ to 10 mm³, from 0.1 mm³ to 100 mm³, from 0.1 mm³ to 1000 mm³, from 0.1 mm³ to 10000 mm³, from 0.1 mm³ to 25000 mm³, from 1 mm³ to 10 mm³, from 1 mm³ to 100 mm³, from 1 mm³ to 1000 mm³, from 1 mm³ to 10000 mm³, from 1 mm³ to 25000 mm³, from 1 mm³ to 50000 mm³, from 10 mm³ to 100 mm³, from 10 mm³ to 1000 mm³, from 10 mm³ to 10000 mm³, from 10 mm³ to 25000 mm³, from 10 mm³ to 50000 mm³, from 100 mm³ to 1000 mm³, from 100 mm³ to 10000 mm³, from 100 mm³ to 25000 mm³, from 100 mm³ to 50000 mm³, from 1000 mm³ to 10000 mm³, from 1000 mm³ to 25000 mm³, from 1000 mm³ to 50000 mm³, from 10000 mm³ to 25000 mm³, from 10000 mm³ to 50000 mm³, or from 25000 mm³ to 50000 mm³.

To permit visualization of objects (e.g., particles such as cells) in the reservoir of the device (e.g., by a microscope) the devices, described herein, may further comprise a substantially planar transparent substrate and/or one or more substantially planar transparent walls or surfaces. This substantially planar transparent substrate, surface, or wall can be, for example, glass or plastic, that permits observation into the reservoir by microscopy so that at least one particle in the reservoir can be observed. In one example, the transparent substrate, surface or wall has a thickness of 0.05 mm to 1 mm. In some cases, the transparent substrate or surface may be a microscope cover slip, or similar component. Microscope coverslips are widely available in several standard thicknesses that are identified by numbers, as follows: No. 0-0.085 to 0.13 mm thick, No. 1-0.13 to 0.16 mm thick, No. 1.5-0.16 to 0.19 mm thick, No. 2-0.19 to 0.23 mm thick, No. 3-0.25 to 0.35 mm thick, No. 4-0.43 to 0.64 mm thick, any one of which may be used as a transparent substrate, surface or wall, depending on the device, microscope, and/or particle size.

The device comprises a first pair of surface acoustic wave (SAW) generators having a first variable power/frequency SAW generator 1 and a second variable power/frequency SAW generator 2 that are disposed on the substrate and on opposing sides of the reservoir 12 to generate surface acoustic waves within the reservoir having a first SAW path 9. As used herein, the term “variable power/frequency SAW generator” refers to a SAW generator (e.g., an IDT or S-IDT) in which the frequency and/or phase is adjustable, and in which the power is adjustable. The first variable power/frequency SAW generator 1 vibrates the substrate in response to a first input signal, which is delivered to the first variable power/frequency SAW generator 1 via a connection 5. The second variable power/frequency SAW generator 2 vibrates the surface in response to a second input signal, which is delivered to the second variable power/frequency SAW generator 2 via a connection 6. The first variable power/frequency SAW generator 1 and the second variable power/frequency SAW generator 2 generate surface acoustic waves within the reservoir having a first SAW path 9.

The device comprises a second pair of surface acoustic wave (SAW) generators having a third variable power/frequency SAW generator 3 and a fourth variable power/frequency SAW generator 4 that are disposed on the substrate and on opposing sides of the reservoir 12 to generate surface acoustic waves within the reservoir having a second SAW path 10. The third variable power/frequency SAW generator 3 vibrates the surface in response to a third input signal, which is delivered to the third variable power/frequency SAW generator 3 via a connection 7. The fourth variable power/frequency SAW generator 4 vibrates the substrate in response to a fourth input signal, which is delivered to the fourth variable power/frequency SAW generator 4 via a connection 8. The third variable power/frequency SAW generator 3 and the fourth variable power/frequency SAW generator 4 generate surface acoustic waves within the reservoir having a second SAW path 10.

The first pair of SAW generators and the second pair of SAW generators are configured on the substrate such that the first SAW path 9 and the second SAW path 10, are different. That is to say, that the pairs of SAW generators are configured such that, in some embodiments, the first SAW path and the second SAW path intersect in the reservoir. In the embodiment depicted, the first SAW path 9 is disposed at a right angle relative to the second SAW path 10. In some embodiments, the first SAW path is disposed at an oblique angle relative to the second SAW path. In some In some embodiments, the first SAW path is disposed at an angle ranging from 1 degree to 90 degrees relative to the second SAW path. In some In some embodiments, the first SAW path is disposed at an angle ranging from 1 degree to 5 degrees, from 1 degree to 10 degrees, from 1 degree to 20 degrees, from 1 degree to 40 degrees, from 1 degree to 60 degrees, from 1 degree to 80 degrees, from 5 degrees to 10 degrees, from 5 degrees to 20 degrees, from 5 degrees to 40 degrees, from 5 degrees to 60 degrees, from 5 degrees to 80 degrees, from 5 degrees to 90 degrees, from 10 degrees to 20 degrees, from 10 degrees to 40 degrees, from 10 degrees to 60 degrees, from 10 degrees to 80 degrees, from 10 degrees to 90 degrees, from 20 degrees to 40 degrees, from 20 degrees to 60 degrees, from 20 degrees to 80 degrees, from 20 degrees to 90 degrees, from 40 degrees to 60 degrees, from 40 degrees to 80 degrees, from 40 degrees to 90 degrees, from 60 degrees to 80 degrees, from 60 degrees to 90 degrees, or from 80 degrees to 90 degrees relative to the second SAW path.

In some embodiments, the device comprises a particle 18 in the reservoir, which may be moved within the reservoir along three mutually orthogonal axes, depicted as solid arrows, labeled X and Y or dotted arrows, labeled Z. For example, the particle 18 may move along a first SAW path in a direction depicted by the X arrows. In some embodiments, the particle 18 may move along a second SAW path in a direction depicted by the Y arrows. In some embodiments, the particle 18 may move along a path orthogonal to the first SAW path and the second SAW path, in a direction depicted by the Z arrows. In some embodiments, movement of the particle in a direction depicted by the Z arrows may be controlled by tuning the input acoustic power to one or more of the first, second, third, and/or fourth variable power/frequency SAW generators.

In some embodiments, the device comprises an electronic control circuit 15 that is connected (e.g., by one or more wires) to the first 1, second 2, third 3, and/or fourth 4 variable power/frequency SAW generators. In some embodiments, the first 1, second 2, third 3, and/or fourth 4 variable power/frequency SAW generators are connected to the control circuit via connections 5, 6, 7, and 8 respectively. In some embodiments, the electronic control circuit is configured to independently and/or simultaneously vary the input power, the frequency and/or the phase of each of a first, a second, a third, and/or a fourth input signal to each of the first 1, the second 2, the third 3, and the fourth 4 variable power/frequency SAW generators. In some embodiments, the electronic control circuit 15 comprises one or more signal generators.

In some embodiments, the device comprises one or more signal generators. In some embodiments, the one or more signal generators are part of an electronic control circuit. In some embodiments, the device comprises 1, 2, 3, 4, 5, 6, 7, 8, 9, or 10 signal generators. In some embodiments, the device comprises 2 signal generators. In some embodiments, the device comprises 4 signal generators. In some embodiments, the first pair of SAW generators is connected to a first radio-frequency (RF) signal generator and the second pair of SAW generators is connected to a second RF signal generator. In some embodiments, each SAW generator is connected to an independent RF signal generator. In some embodiments, the RF signal generators comprise one or more amplifiers. In some embodiments, one or more RF signal generators comprise 1, 2, 3, 4, 5, 6, 7, 8, 9 or 10 amplifiers. It should be appreciated that the RF signal generators provided herein may be part of an electronic control circuit 15, which can be used to independently and/or simultaneously vary an input power, a frequency and/or a phase of one or more input signals that are delivered to one or more SAW generators.

In some embodiments, the electronic control circuit is configured to vary the input power to one or more surface acoustic wave generators. In some embodiments, the electronic control circuit is configured to vary the input power to one or more surface acoustic wave generators from 0.1 mW to 2000 mW. In some embodiments, the electronic control circuit is configured to vary the input power to one or more surface acoustic wave generators from 0.1 mW to 1 mW, from 0.1 mW to 10 mW, from 0.1 mW to 100 mW, from 0.1 mW to 1000 mW, from 1 mW to 10 mW, from 1 mW to 100 mW, from 1 mW to 1000 mW, from 1 mW to 2000 mW, from 10 mW to 100 mW, from 10 mW to 1000 mW, from 10 mW to 2000 mW, 100 mW to 1000 mW, from 100 mW to 2000 mW, or from 1000 mW to 2000 mW.

In some embodiments, the electronic control circuit is configured to vary the frequency to one or more surface acoustic wave generators. In some embodiments, the electronic control circuit is configured to vary the frequency to one or more surface acoustic wave generators from 1 MHz to 1000 MHz. In some embodiments, the electronic control circuit is configured to vary the frequency to one or more surface acoustic wave generators from 1 MHz to 5 MHz, from 1 MHz to 10 MHz, from 1 MHz to 20 MHz, from 1 MHz to 30 MHz, from 1 MHz to 50 MHz, from 1 MHz to 100 MHz, from 1 MHz to 500 MHz, from 5 MHz to 10 MHz, from 5 MHz to 20 MHz, from 5 MHz to 30 MHz, from 5 MHz to 50 MHz, from 5 MHz to 100 MHz, from 5 MHz to 500 MHz, from 5 MHz to 1000 MHz, from 10 MHz to 20 MHz, from 10 MHz to 30 MHz, from 10 MHz to 50 MHz, from 10 MHz to 100 MHz, from 10 MHz to 500 MHz, from 10 MHz to 1000 MHz, from 20 MHz to 30 MHz, from 20 MHz to 50 MHz, from 20 MHz to 100 MHz, from 20 MHz to 500 MHz, from 20 MHz to 1000 MHz, from 30 MHz to 50 MHz, from 30 MHz to 100 MHz, from 30 MHz to 500 MHz, from 30 MHz to 1000 MHz, from 50 MHz to 100 MHz, from 50 MHz to 500 MHz, from 50 MHz to 1000 MHz, from 100 MHz to 500 MHz, from 100 MHz to 1000 MHz, or from 500 MHz to 1000 MHz.

In some embodiments, the electronic control circuit is configured to vary the phase to one or more surface acoustic wave generators. In some embodiments, the electronic control circuit is configured to vary the phase to one or more surface acoustic wave generators by at least 0.1 degree or by at least 1 degree. In some embodiments, the electronic control circuit is configured to vary the phase to one or more surface acoustic wave generators by at least 0.1 degree to 20 degrees. In some embodiments, the electronic control circuit is configured to vary the phase to one or more surface acoustic wave generators by at least 1 degree, 2 degrees, 5 degrees, 10 degrees, 15 degrees, or 20 degrees. In some embodiments, the electronic control circuit is configured to vary the phase to one or more surface acoustic wave generators by 1 degree to 2 degrees, by 1 degree to 5 degrees, by 1 degree to 10 degrees, by 1 degree to 15 degrees, by 1 degree to 20 degrees, by 2 degrees to 5 degrees, by 2 degrees to 10 degrees, by 2 degrees to 15 degrees, by 2 degrees to 20 degrees, by 5 degrees to 10 degrees, by 5 degrees to 15 degrees, by 5 degrees to 20 degrees, by 10 degrees to 15 degrees, by 10 degrees to 20 degrees, or by 15 degrees to 20 degrees.

In some embodiments, the reservoir comprises an inlet port 13 and/or an outlet port 14. In the embodiment depicted, the inlet port and the outlet port are on opposite walls of the reservoir 12. It should be appreciated that the reservoir may have one or more inlet ports and/or one or more outlet ports. In some embodiments, the reservoir has 1, 2, 3, 4, 5, 6, 7, 8, 9 or 10 inlet ports. In some embodiments, the reservoir has 1, 2, 3, 4, 5, 6, 7, 8, 9 or 10 outlet ports. In some embodiments one or more of the walls of the reservoir have one or more inlet and/or outlet ports. In some embodiments, the top surface of the reservoir has one or more inlet and/or outlet ports. In some embodiments, the substrate of the reservoir has one or more inlet and/or outlet ports.

In some embodiments, the first 1, second 2, third 3, and/or fourth 4 variable power/frequency SAW generators are interdigital transducers. The variable power/frequency interdigital transducers (IDTs) may include first and second integrated electrodes supported by the substrate, driven by an input signal applied between the electrodes by an electronic control circuit 15. In some embodiments, the finger spacing between neighboring electrode fingers varies as a function of position on the substrate, so that the IDT may have a broad frequency response. A variable power/frequency interdigital transducer (IDT) for use with the present device may have a tunable acoustic output. In some embodiments, a variable power/frequency interdigital transducer (IDT) may have a tunable acoustic output ranging from 0.1 dBM to 40 dBm. In some embodiments, a variable power/frequency interdigital transducer (IDT) for use with the present device may have a tunable acoustic output ranging from 0.1 dBm to 1 dBm, from 0.1 dBm to 2 dBm, from 0.1 dBm to 5 dBm, from 0.1 dBm to 10 dBm, from 0.1 dBm to 20 dBm, from 0.1 dBm to 30 dBm, from 1 dBm to 2 dBm, from 1 dBm to 5 dBm, from 1 dBm to 10 dBm, from 1 dBm to 20 dBm, from 1 dBm to 30 dBm, from 1 dBm to 40dBm, from 2 dBm to 5 dBm, from 2 dBm to 10 dBm, from 2 dBm to 20 dBm, from 2 dBm to 30 dBm, from 2 dBm to 40 dBm, from 5 dBm to 10 dBm, from 5 dBm to 20 dBm, from 5 dBm to 30 dBm, from 5 dBm to 40 dBm, from 10 dBm to 20 dBm, from 10 dBm to 30 dBm, from 10 dBm to 40 dBm, from 20 dBm to 30 dBm, from 20 dBm to 40 dBm, or from 30 dBm to 40 dBm.

A variable power/frequency interdigital transducer (IDT) for use with the present device may have a tunable frequency. In some embodiments, a variable power/frequency interdigital transducer (IDT) may have a tunable frequency ranging from 1 MHz to 100 MHz. In some embodiments, a variable power/frequency interdigital transducer (IDT) may have a tunable frequency ranging from 1 MHz to 5 MHz, from 1 MHz to 10 MHz, from 1 MHz to 20 MHz, from 1 MHz to 50 MHz, from 1 MHz to 80 MHz, from 5 MHz to 10 MHz, from 5 MHz to 20 MHz, from 5 MHz to 50 MHz, from 5 MHz to 80 MHz, from 5 MHz to 100 MHz, from 10 MHz to 20 MHz, from 10 MHz to 50 MHz, from 10 MHz to 80 MHz, from 10 MHz to 100 MHz, from 50 MHz to 80 MHz, from 50 MHz to 100 MHz, or from 80 MHz to 100 MHz.

A variable power/frequency interdigital transducer (IDT) for use with the present device may have a tunable phase. In some embodiments, a variable power/frequency interdigital transducer (IDT) may have a tunable phase ranging from 0.1 to 360 degrees.

In some embodiments, the IDTs provided herein may be segmented IDTs (S-IDTs). Instead of using parallel IDTs, S-IDTs consist of many small sections of parallel IDTs. Each section has a consistent displacement from the previous one in the lateral direction. The function of a S-IDT is to generate many discontinued, independent SAW fields in the reservoir. A non-limiting schematic of a pair of S-IDTs disposed on opposite sides of a reservoir is depicted in FIG. 11. In some embodiments, the number of segments of the segmented interdigital transducer range from 5 to 30. In some embodiments, the length of any of the segments range from 100 μm to 1000 μm. In some embodiments, the segmented interdigital transducer has 15 segments, wherein the length of the segments is 250 μm.

Alternatively or additionally, the devices described herein further contain a heat transfer element, which can maintain the fluid at a predetermined temperature (e.g., a physiologically relevant temperature (e.g., a temperature that would be found in vivo in a healthy or diseased subject or one with a particular condition as provided herein), such as 30° C. to 45° C., preferably 37° C. or 41° C.).

In some embodiments, the electronic control circuit 15 is optionally connected to a computer 17, which may be used to control one or more input signals from the electronic control circuit to one or more of the variable power/frequency SAW generators. In some embodiments, the computer 17 is connected to the control circuit 15 by a connection 16, which may be a wired connection, or a wireless connection. Any of the above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Printing One or More Biological Cells onto a Substrate

Aspects of the disclosure relate to methods for printing one or more biological cells onto a substrate. As used herein, the term “printing” refers to the process of generating spatially-controlled cell patterns using 3D printing technologies, such as the methods and devices provided herein. In some embodiments, methods and devices used to print cells preserve cell function and/or viability. In some embodiments, printing involves dispensing one or more cells onto a biocompatible substrate or scaffold. In some embodiments, printing involves dispensing one or more cells onto a biocompatible substrate or scaffold using a successive layer-by-layer approach to generate tissue-like three-dimensional structures

In some embodiments, printing one or more biological cells onto a substrate includes

a.) providing a reservoir disposed on a substrate, wherein the reservoir comprises one or more biological cells;

b.) generating a first surface acoustic wave (SAW) and a second SAW along a first axis of the substrate, wherein the first SAW and the second SAW are generated from opposite sides of the reservoir;

c.) generating a third SAW and a fourth SAW along a second axis of the substrate, wherein the third SAW and the fourth SAW are generated from opposite sides of the reservoir, and wherein the first axis and the second axis intersect in the reservoir;

d.) manipulating the one or more biological cells in the reservoir to move along the first axis and/or the second axis by varying a frequency and/or a phase of at least one of the first, the second, the third, and/or the fourth SAW; and

e.) manipulating the one or more biological cells in the reservoir to move along an axis that is orthogonal to the first axis and the second axis by varying an acoustic power of at least one of the first, the second, the third, and/or the fourth SAW; and

f.) depositing the one or more cells onto a cell substrate.

Cells may be deposited onto a cell substrate in the reservoir using any of the methods and devices provided herein. For example, a cell may be positioned above a cell substrate (e.g., a substrate that is located at the bottom of the reservoir) at a desired position by moving the cell along the first and/or the second axis. The cell may then be deposited onto a cell substrate (e.g., that is located at the bottom of the reservoir) by adjusting the acoustic power of the first, second, third, and/or fourth SAWs. In some embodiments, a single cell is deposited onto a cell substrate. In some embodiments, more than one cells are deposited onto a cell substrate. In some embodiments, cells are deposited onto a cell substrate one at a time. In some embodiments, a plurality of cells are deposited onto a cell substrate at the same time. It should be appreciated that the methods and devices provided herein allow for the deposition of one or more cells onto a cell substrate with a high degree of accuracy. For example, a cell may be placed within 0.1 μm of a desired location on the cell substrate. In some embodiments, a cell may be placed on top of another cell on a cell substrate. For example, cells may be layered on top of, or beside each other in order to recreate or mimic the 3D structure of one or more tissues. In some embodiments, a cell may be placed within 0.1 μm, 0.2 μm, 0.3 μm, 0.4 μm, 0.5 μm, 0.6 μm, 0.8 μm, 0.9 μm, 1 μm, 1.2 μm, 1.4 μm, 1.6 μm, 1.7 μm, 2 μm, 2.2 μm, 2.4 μm, 2.8 μm, 3 μm, 3.5 μm, 4.0 μm, 4.5 μm, or 5.0 μm, of a desired location on the cell substrate. It should also be appreciated that the methods and devices provided herein are useful for preserving cell viability. In some embodiments, at least 50% of the cells deposited onto a cell substrate are viable. In some embodiments, at least 50%, 55%, 60%, 65%, 70%, 75%, 80%, 85%, 90%, 95%, 98%, 99% or 100% of the cells deposited onto a cell substrate are viable. Viability of cells may be tested by any suitable means. For example, cell viability may be tested using a cytolysis or membrane leakage test (e.g., trypan blue, propidium iodine, or 7-aminoactinomycin D), a mitochondrial assay, a caspase assay, a functional assay, or a genomic or proteomic assay.

In some embodiments, one or more cells are deposited onto a cell substrate. A “cell substrate” as used herein, refers to any substrate that a cell may be attached and/or adhered to. For example, a “cell substrate may be the substrate that the reservoir is disposed on. In some embodiments, the cell substrate is a second substrate that is disposed in the reservoir. The cell substrate may be located at any position in the reservoir. In some embodiments, the second substrate is located at the bottom of the reservoir. In some embodiments, the second substrate is located at the top of the reservoir. In some embodiments, the cell substrate is a wall of the reservoir. For example, the second substrate may be a bottom wall, a top wall, or any side wall of the reservoir.

In some embodiments, the second substrate comprises a scaffold. As used herein a “scaffold” refers to a supporting framework. In some embodiments, the scaffold comprises synthetic material. A scaffold that comprises synthetic material may be referred to as a synthetic scaffold. In some embodiments, the scaffold comprises biological material. A scaffold that comprises biological material may be referred to as a biological scaffold. A “biological material” refers to any material that is obtained or derived from a cell or organism. In some embodiments, a biological material is a cell, organism, protein, nucleic acid, organelle, or an extra cellular matrix. A variety of different scaffolds can be used for seeding, growing, supporting, or maintaining cells, tissues, and organs as described herein. A scaffold can have any suitable shape and may depend on the particular tissue and/or organ to be grown. For example, the scaffold may be substantially tubular, substantially cylindrical, substantially spherical, substantially planar, substantially ellipsoidal, disk-like, sheet-like, or irregularly shaped. The scaffold can also have branching structures, e.g., to mimic arteries, veins, or other vessels. In certain embodiments, at least a portion of the scaffold is hollow.

Scaffolds may be formed of natural and/or artificial materials. Materials used to form scaffolds may be biocompatible, and can include synthetic or natural polymers, inorganic materials (e.g., ceramics, glass, hydroxyapatite and calcium carbonate), composites of inorganic materials with polymers, and gels. All or a portion of a scaffold may be formed in a material that is non-biodegradable or biodegradable (i.e., via hydrolysis or enzymatic cleavage). In some embodiments, biodegradable polyesters such as polylactide, polyglycolide, and other alpha-hydroxy acids can be used to form scaffold. By varying the monomer ratios, for example, in lactide/glycolide copolymers, physical properties and degradation times of the scaffold can be varied. For instance, poly-L-lactic acid (PLLA) and poly-glycolic acid (PGA) exhibit a high degree of crystallinity and degrade relatively slowly, while copolymers of PLLA and PGA, PLGAs, are amorphous and rapidly degraded. A portion of a scaffold that is biodegradable may, in some embodiments, degrade during the growth of cells, tissues and/or organs in the reservoir. In other embodiments, degradation may take place after implanting the tissue or organ in a recipient.

Optionally, surface properties of a cell substrate can be modified by various techniques. For example, in some cases, surfaces of a cell substrate (e.g., a scaffold) can be modified by coating and/or printing an additive proximate the cell substrate. Surfaces of a cell substrate may be modified with additives such as proteins and/or other suitable surface-modifying substances. For example, collagen, fibronectin, an RGD peptide, and/or other extracellular matrix (ECM) proteins or growth factors can be coated onto the cell substrate, e.g., to elicit an appropriate biological response from cells, including cell attachment, migration, proliferation, differentiation, and gene expression. Cells can then be seeded onto surfaces of a cell substrate using any of the methods and/or devices provided herein.

In some embodiments, any of the scaffolds provided herein may contain biological and/or artificial material (e.g., biological and/or artificial polymers). In some embodiments, the scaffold may consist entirely of biological material (e.g., one or more biological polymers). In some embodiments, the scaffold may consist entirely of artificial material (e.g., one or more synthetic polymers). In some embodiments, a scaffold may include a mixture of one or more biological materials and/or one or more artificial materials. In some embodiments, the materials are shaped (e.g., on a template, in a mold, or using any other suitable shaping technique, or any combination thereof) to have a suitable conformation (e.g., a three-dimensional conformation) and size (e.g., volume of cells, diameter and/or length of blood vessels, airways, and/or other ducts, etc.).

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1

### Three-dimensional Manipulation of Single Cells Using Surface Acoustic Waves

Presented here are three-dimensional (3D) acoustic tweezers, which can trap and manipulate single cells and particles along three mutually orthogonal axes of motion by recourse to surface acoustic waves. 3D acoustic tweezers were used to pick up single cells, or entire cell assemblies, and deliver them to desired locations to create two- and three-dimensional cell patterns, or print the cells into complex arrays. This technology is thus shown to offer better performance over prior cell manipulation techniques in terms of both accurate and precise motion in a noninvasive, label-free, and contactless manner. This method offers the ability to accurately print 3D multicellular architectures for applications in bio-manufacturing, tissue engineering, regenerative medicine, neuroscience, and cancer metastasis research.

“Acoustic tweezers”, which manipulate biological specimens using sound waves, offer several unique advantages (12, 13) in comparison to other techniques. First, acoustic tweezers technology is the only active cell-manipulation method employing gentle mechanical vibrations that do not alter cell characteristics. Acoustic vibrations create a pressure gradient in the medium to move suspended micro-objects and cells, thereby resulting in a contamination-free, contact-less, and label-free method for cell manipulation. Sound waves are preferred for cell manipulation for at least the following reasons: 1) Cells maintain their native state (e.g., shape, size, reflective index, charge, or polarity) in the absence of surface modification or labelling; 2) Cells can remain in their original culture medium or extracellular matrix gel solution. Furthermore, acoustic tweezers are safe tools for biological manipulation. Acoustic tweezers involving sound waves have a power intensity that is approximately 10 million times lower than that of optical tweezers. Therefore, acoustic tweezers have minimal impact on cell viability and function. Moreover, acoustic tweezers operate at a power intensity and frequency similar to the widely-used medical ultrasound method that is accepted as a safe technique for sensitive clinical applications such as imaging of a fetus in the mother's womb. Finally, an acoustic tweezers platform can be constructed as a single, integrated micro-device without any moving parts or complicated setup procedures. This feature offers additional advantages for ease of use and versatility.

Thus far, sound waves have been demonstrated to successfully perform many micro-scale functions such as the separation, alignment, enrichment, patterning, and transportation of cells and micro-particles (12-17). None of these acoustic approaches, however, has hitherto demonstrated controlled 3D manipulation of single cells. This is mainly due to the limited understanding of the relationship between a 3D acoustic field and the induced acoustic streaming. Employing two-dimensional (2D) acoustic waves often results in insufficient control of a single cell in 3D space. Described herein is a standing surface acoustic wave (SSAW)-based technique that is able to create and independently manipulate an array of stable 3D trapping nodes.

Illustrated herein is the relation between the acoustic vibrations, the acoustic field produced by SSAWs, and the resulting streaming in a microfluidic chamber by recourse to both modeling and controlled experimental validation. Unlike Rayleigh streaming in bulk acoustic wave devices, the unique acoustic streaming (i.e., streaming motion caused by acoustic oscillation) pattern in a SSAW device determines how objects are lifted up from the substrate surface. By regulating the 3D distributed acoustic field and acoustic streaming, induced by two superimposed pairs of orthogonally placed SSAWs, an array of 3D trapping nodes in a microfluidic chamber was achieved. By independently tuning the relative phase angle of each SSAW or by varying the input power, the position of these 3D trapping nodes can be precisely controlled in a 3D environment. Three-dimensional trapping and three-axis manipulation of single micro-particles and single biological cells is demonstrated using this concept. Finally, how a 3D acoustic tweezers technique can be used for printing with live cells, and for producing prescribed cell culture patterns is illustrated herein.

### Working Mechanism of 3D Acoustic Tweezers

In order to manipulate suspended objects along three orthogonal axes with surface acoustic waves (SAWs), it is necessary to form 3D trapping nodes and to move these nodes precisely in three dimensions. To achieve this objective, a 2D displacement field on a lithium niobate (LiNbO₃) piezoelectric substrate by superimposing two mutually orthogonal pairs of interdigital transducers (IDTs) that produce SSAWs was created. The propagation of these waves through a microfluidic chamber (e.g., reservoir) produces an acoustic field distributed in three dimensions and induces acoustic streaming which creates stable 3D trapping nodes within the fluid-filled chamber. The positions of these 3D trapping nodes were precisely manipulated in the transverse (X), longitudinal (Y), or vertical (Z) directions by adjusting the phase angle of each individual IDT pair (X- or Y-axis motion control) or the input acoustic power (Z-axis motion control), respectively.

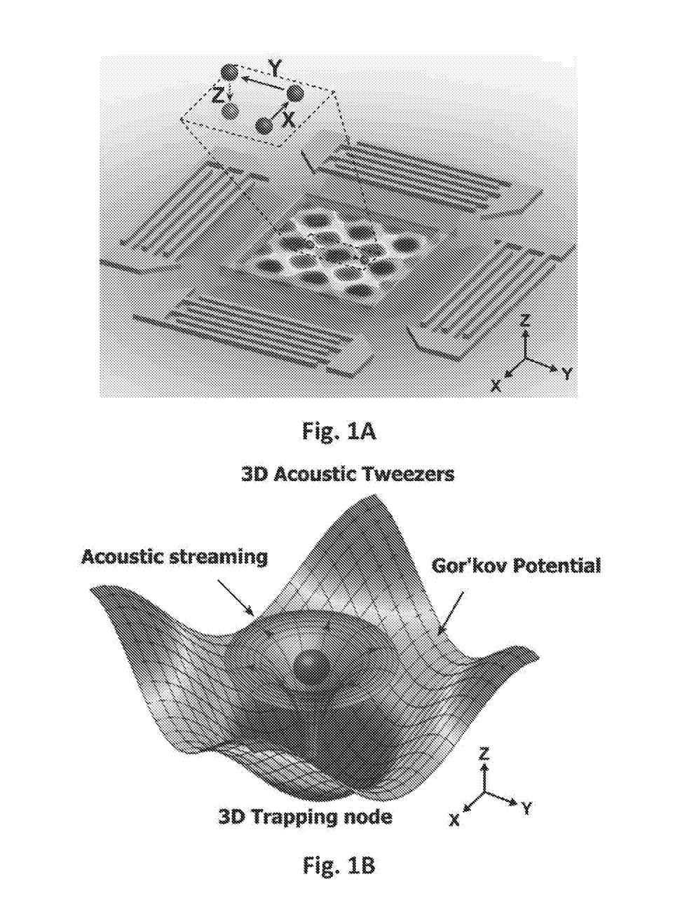

Two orthogonal SSAWs were employed to perform particle/cell manipulation in a microfluidic chamber. Two pairs of IDTs were deposited onto a 128° YX-cut LiNbO₃ substrate, which were positioned along the X and Y axes. The IDTs were made up of 40 pairs of electrodes with a 75 μm width of each finger electrode and a 75 μm spacing between fingers, and a 1 cm aperture. A polydimethylsiloxane (PDMS) layer with a 1.8 mm×1.8 mm×100 μm fluidic chamber was bonded to the substrate, at the center of the two orthogonal pairs of IDTs. FIG. 1A shows a schematic diagram of the device. Each pair of IDTs was individually connected to a double-channel radio-frequency (RF) signal generator and two amplifiers, which generated SSAWs with different frequencies and independent SSAW phase angle control. Once the pairs of IDTs were activated, a 2D displacement field (including both longitudinal and transverse vibrations) was produced on the surface of the LiNbO₃ substrate (18). The acoustic waves induced by these surface vibrations propagated in the fluid, reflected by the chamber walls, and established a 3D, differential Gor'kov potential field (19). Meanwhile, these surface vibrations also induced 3D acoustic streaming in the microfluidic chamber. The interaction of the fluidic and acoustic fields produced 3D trapping nodes within the chamber (FIG. 1B). Along the vertical direction, suspended micro-objects were pushed and levitated to a stable trapping node due to the competitive interaction of the acoustic radiation force, the gravitational force, the buoyancy force, and the Stokes drag force induced by acoustic streaming. After increasing the input acoustic power, the vertical trapping position was raised due to a rebalancing of these forces. Along the horizontal plane (X-Y plane), the objects were pushed toward the center of the 3D trapping node. These trapping positions can be independently manipulated along the transverse (X) or longitudinal (Y) directions by relocating the 2D displacements via changing the input phase angle. As a result, micro-objects were trapped into a 3D node and manipulated along three axes within a microfluidic chamber (FIG. 1A).

### SAW Vibrations Induce 3D Acoustic and Fluidic Fields

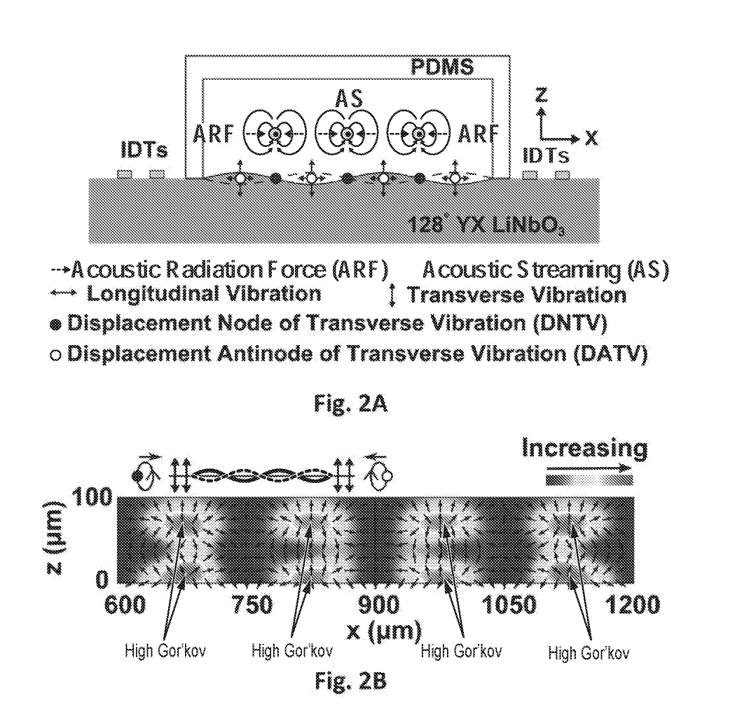

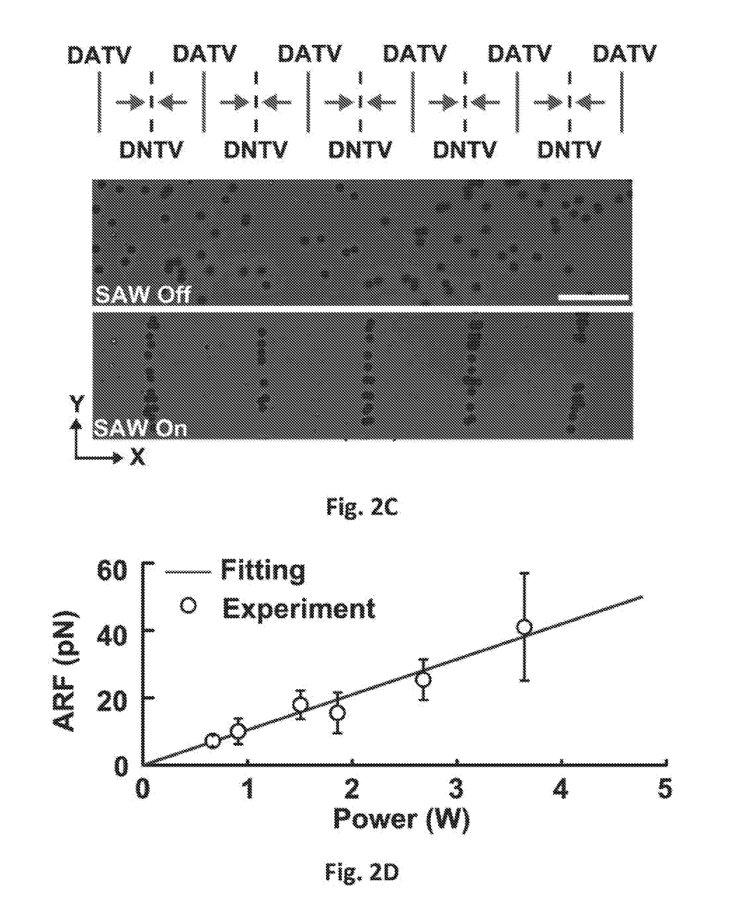

In order to create a 3D trapping node in a microfluidic chamber, the mechanism by which SSAWs manipulate objects within such a chamber must be understood. A simple acoustic tweezers device, consisting of a PDMS chamber and a pair of IDTs (positioned along the X axis of a 128° YX LiNbO₃ substrate), was used to investigate this mechanism. Two sets of SAWs, travelling towards each other, were produced after applying a RF signal to the IDTs. A SSAW was formed via the superposition of these SAWs. This type of resulting wave is considered a Rayleigh wave. These waves confine most of the energy to the surface due to the exponential decay of their amplitude with the depth of the substrate. In addition, these waves include both longitudinal and transverse vibrations with a phase lag on the substrate. Once the waves interfere with the liquid in the microfluidic chamber, periodically distributed vibrations are created which lead to periodically distributed acoustic fields and acoustic streaming in the microfluidic chamber (FIG. 2A). A numerical model that could account for the Gor'kov potential and acoustic streaming in a lateral plane (X-Z plane) as well as the acoustic radiation force acting on suspended particles was developed. The model considers the effects of the transverse and longitudinal vibrations on the liquid, and the acoustic reflection and transmission at the interface between the fluid and PDMS. A detailed description of the numerical model can be found under the Materials and Methods,

### Theoretical Framework and Model Setup Heading Herein.

The periodically distributed transverse vibrations are considered as the primary perturbation source for generating an acoustic field in a liquid chamber, while the longitudinal vibrations decay very fast and have little impact on the acoustic field. FIG. 2B predicts the distribution of the Gor'kov potential along the X-Z plane. The regions of maximum Gor'kov potential (in red color), known as pressure antinodes (ANs), are located atop places with a displacement antinode of the transverse vibrations (DATVs), whereas minimum regions (in blue color), known as pressure nodes (PNs), occur atop a displacement node of transverse vibrations (DNTVs). The distribution of PNs and ANs coincide with the location of DNTVs and DATVs on the substrate's surface; therefore the distance between adjacent PNs or ANs is half-wavelength of a SAW. Due to the gradient of the Gor'kov potential, an acoustic radiation force was generated to push the suspended cells or micro-particles from ANs to PNs. The experimental results show that all the suspended 10.1 μm diameter polystyrene particles were pushed to the parallel PNs by the acoustic radiation force (FIG. 2C). The dependence of the acoustic radiation force on input power was investigated and the results were plotted (see FIG. 2D, Materials and Methods, Data analysis).

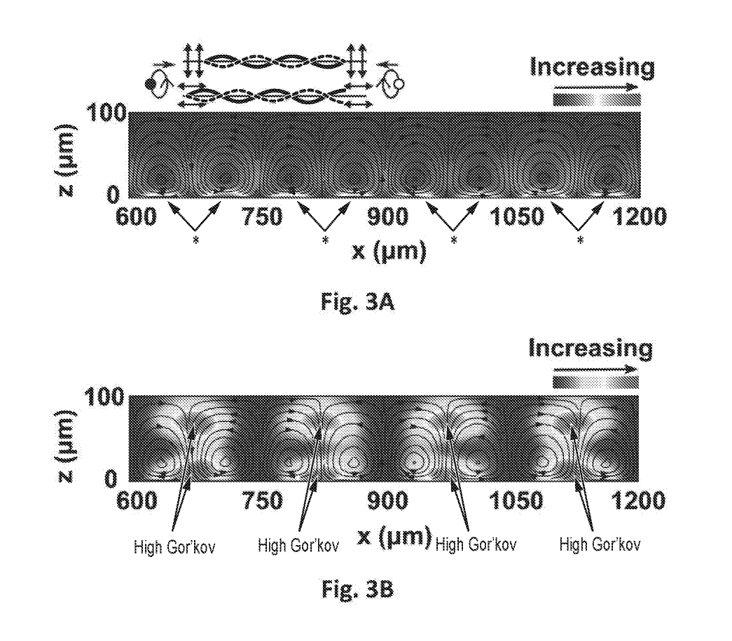

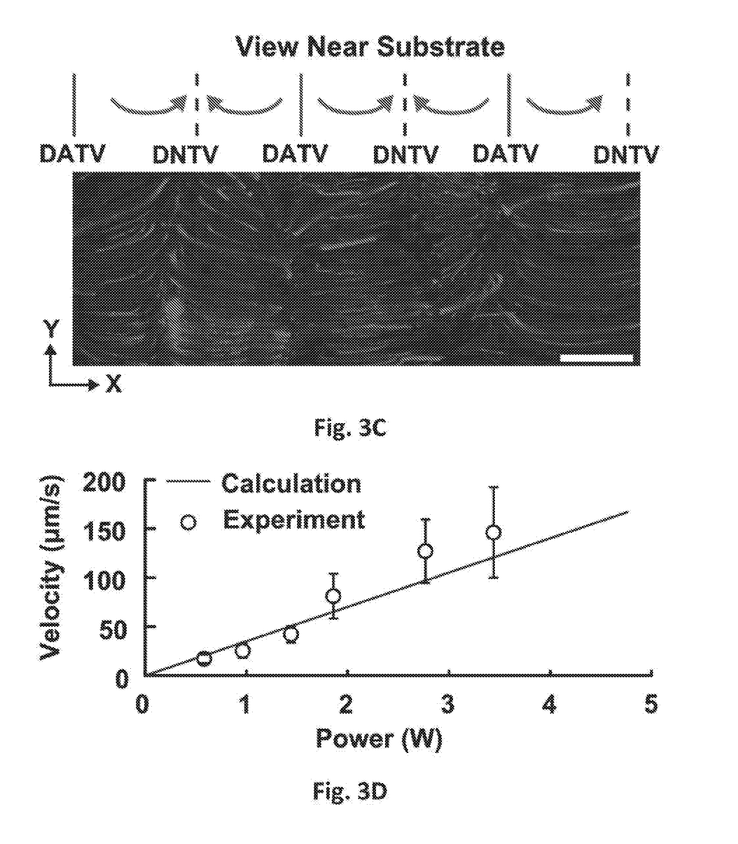

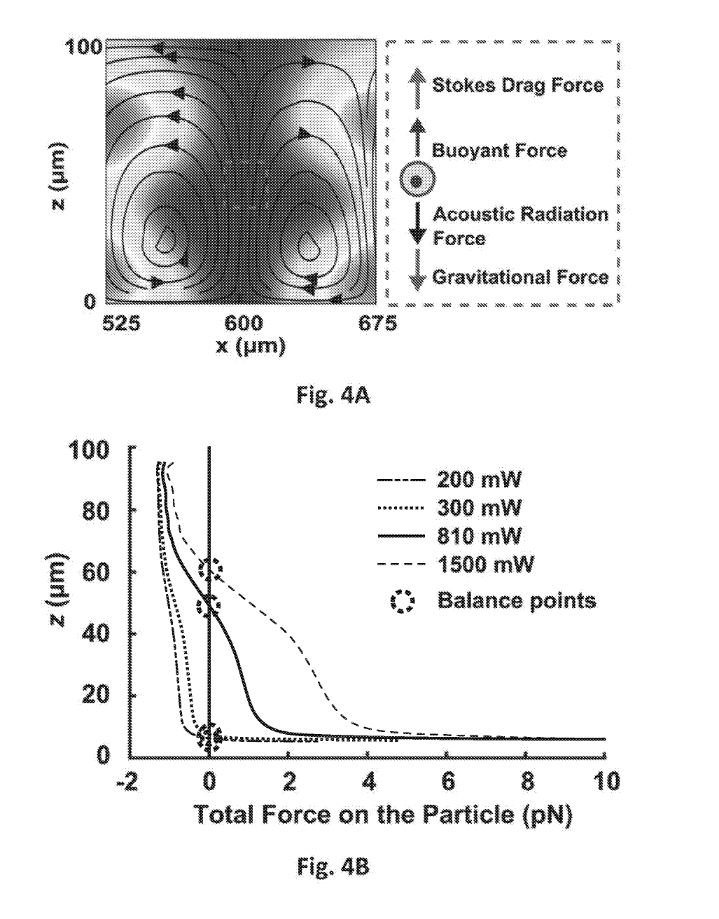

In addition to the aforementioned acoustic field, the vibrations also induced acoustic streaming. Both the transverse and longitudinal vibrations attenuate in a thin boundary layer close to the substrate and result in a particular streaming pattern in the microfluidic chamber (FIG. 2A). Numerical results describe the streaming vortices and the periodic distribution of the acoustic streaming in the X-Z plane, as shown in FIG. 3A. The streaming flows rise up from DNTVs on the substrate, rotate clockwise or counter-clockwise to the two nearby DATVs, and then rise up again from the original DNTVs. In contrast to Rayleigh streaming (18, 20) which is driven by standing bulk acoustic waves, this streaming, which is induced by SSAWs, has a reversed direction with respect to the streaming vortices. However, they share the same spatial distribution with four vortices in one wavelength. This particular streaming plays an important role in the vertical manipulation of micro-particles/cells. FIG. 3B plots the numerical streaming pattern over Gor'kov potential in the X-Z plane. The streaming flows rise vertically from DNTVs on the substrate towards the PNs where particles are trapped.

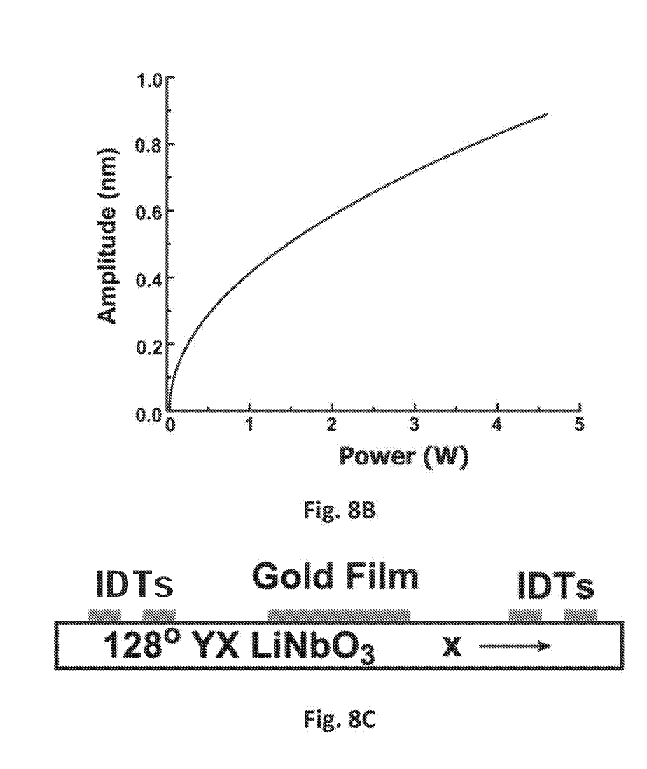

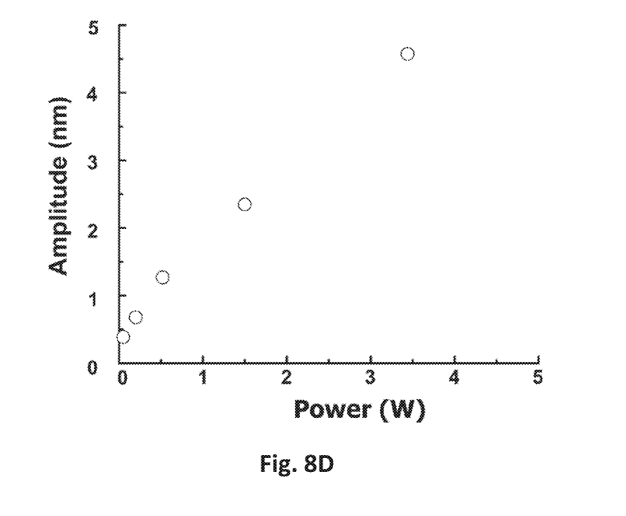

To validate the modeling prediction, an experiment was performed to investigate SSAW-induced acoustic streaming within the microfluidic chamber. The experiment was started by pre-marking the location of DNs by patterning 10.1 μm polystyrene particles into parallel lines. Then, the focal plane of the microscope was fixed near the substrate. Once the SSAW was applied, the 1 μm fluorescent particles, used as the markers to trace streaming lines, flowed up from the DNTVs, defocused, and then flowed down to the neighboring DATVs in the X-Y plane. A time series of frames, captured from the movie, was superposed to show the streaming pattern (FIG. 3C). The magnitude of streaming velocity increases linearly with the input power (see FIG. 3D, Materials and Methods, Data analysis). Based on the quantitative values of the acoustic radiation force and acoustic streaming, the quantitative relationship between the input powers and the amplitudes of the transverse vibrations (˜several nm) on the substrate was calculated (see FIGS. 8A to 8D and Materials and Methods, Calibration of vibration amplitude).

### Manipulation in the Vertical Direction

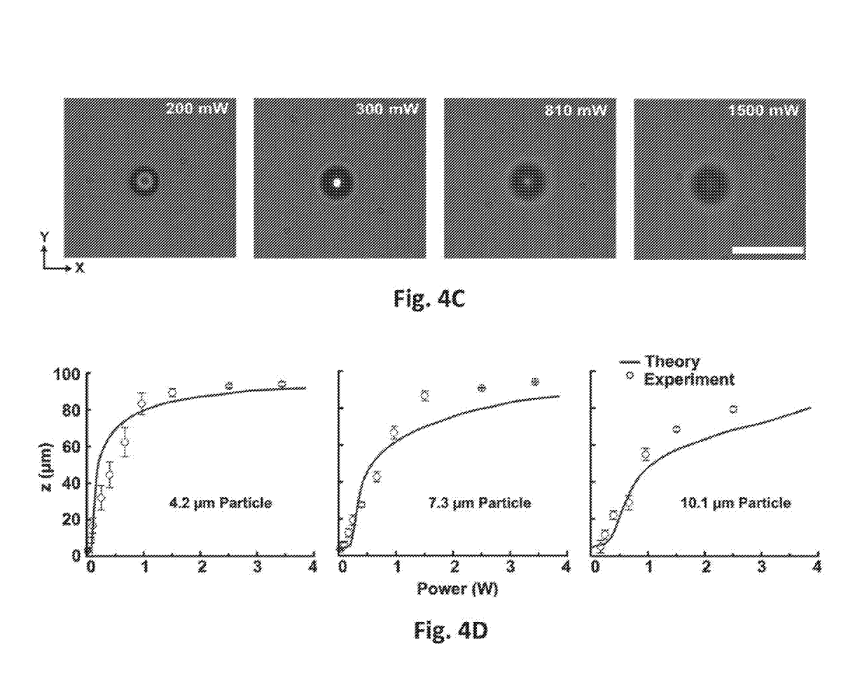

Based on the model of the vertical distribution of the acoustic and fluidic fields induced by a SSAW, the vertical manipulation of trapped objects were explored. Using the aforementioned device with one SSAW, the forces acting on 10.1 μm polystyrene particles were examined in detail. Along the horizontal plane (X-Y plane), the suspended particles experience a greater acoustic radiation force than the Stokes drag force (induced by the acoustic streaming) causing the particles to be pushed toward the PNs. Along the vertical direction, the particle experiences acoustic radiation force, Stokes drag force, gravitational force, and buoyancy force as shown in FIG. 4A. Due to the minimal Gor'kov potential and small gradient within the PNs, the acoustic radiation force in the PNs is comparable to the opposing forces. Once these forces are balanced, the suspended object will be trapped in a stable, vertical position. By adjusting the vibration amplitude along the substrate through tuning the input power of the SAWs, the acoustic radiation force and the Stokes drag force along the Z direction can be regulated accordingly. As a result, the trapped object will move to a new stable position along the Z axis.

In addition, the forces acting on a single 10.1 μm polystyrene particle along the vertical direction are quantitatively investigated by the model (See Materials and Methods, Theoretical framework and model setup). FIG. 4B shows the total force acting on the particle along the Z direction in the chamber (x=600 μm) as a function of the input power (FIG. 4B). The stable trapping positions, where the net force is zero, are marked with blue circles. The trap position rises as the input power increases. To validate the model, additional experiments were conducted in the vertical direction of the chamber. By applying SSAW with a frequency of 13 MHz and an input power of 200 mW, the particles were levitated to the PNs. A single trapped particle can be levitated toward the top of the microfluidic chamber by increasing the input power from 200 mW to 1500 mW (FIG. 4C). The focal plane of the microscope was kept at the same position in FIG. 4C; therefore, defocusing indicated that a single particle was levitated vertically as the input power increased. The absolute position of the trapped particle in the vertical direction was calibrated to a reference plane and could be readily obtained by measuring the change in focal planes while tracking the particle. With this method, it was quantitatively characterized that the vertical trapping position of the particle as a function of input power. FIG. 4D shows the experimental results of the trapping position of single particles with a diameter of 4.2 μm, 7.3 μm, and 10.1 μm, respectively, under different input powers (FIG. 4D). The data sets shown in FIG. 4D were obtained by averaging 10 repeated experimental measurements. The model predictions of the vertical position versus input power are seen to match the experimental results. This method can thus levitate single particles to any vertical position within the chamber.

### 3D Trapping and Manipulation

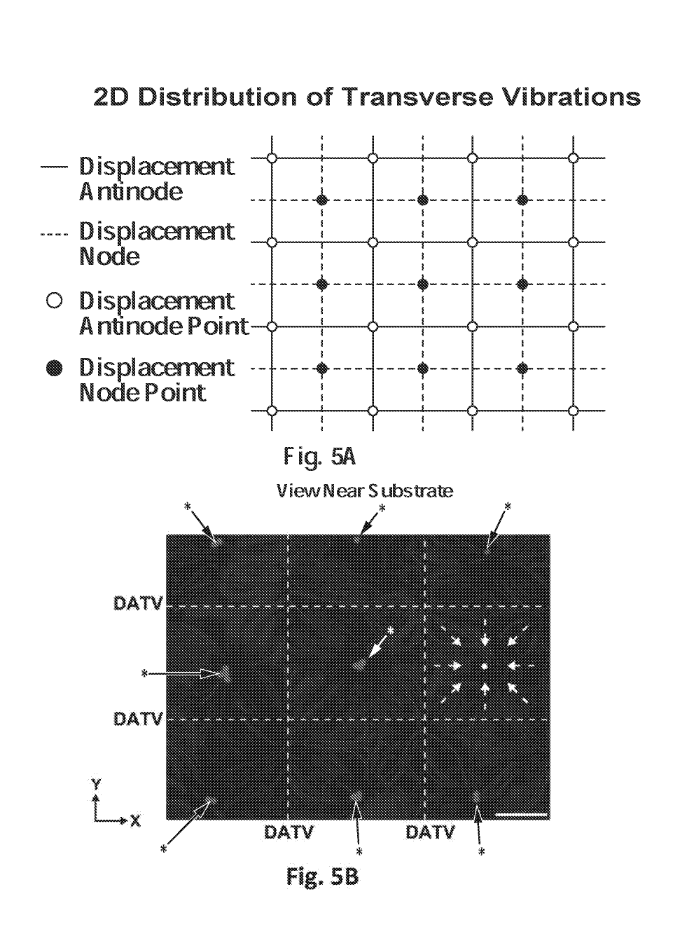

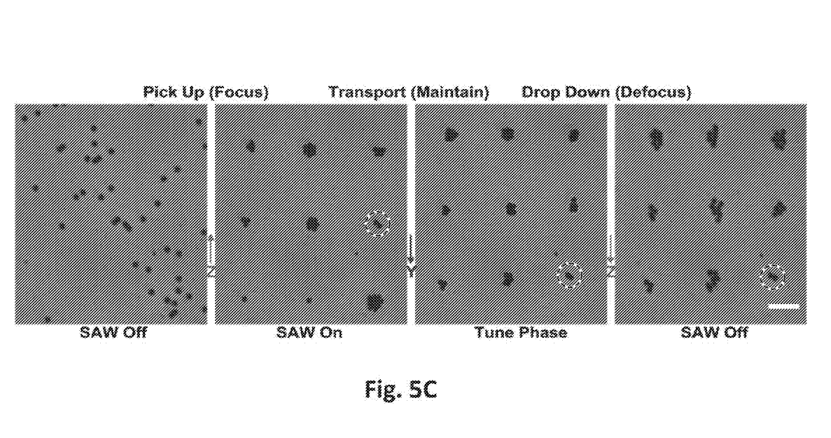

With an understanding of the actuation mechanism for a one dimensional (1D) SSAW, 3D trapping and manipulation was investigated by employing orthogonally-arranged 2D SSAWs. FIG. 5A illustrates the 2D distribution of transverse vibrations from the 2D SSAWs on a substrate: the interaction of DATVs (blue lines) formed node points (blue filled circles) with minimum Gor'kov potentials, and the interaction of DNTVs (red lines) formed antinode points (red filled circles) with maximum Gor'kov potentials. Vibrations from 2D SSAWs exhibit characteristics similar to those of 1D SSAW, and can induce acoustic fields and streaming into the liquid of the microfluidic chamber. The interaction of these two fields forms an array of 3D trapping nodes in the microfluidic chamber with each node superposed on each displacement node of the transverse vibrations.

Next, the acoustic tweezers device with two orthogonal pairs of IDTs was employed to experimentally demonstrate 3D trapping and manipulation. By applying two different RF signals (13 MHz/810 mW and 12 MHz/810 mW) to the two pairs of IDTs (along the X and Y axes, respectively), 3D trapping was achieved as shown in FIG. 5B. The square trapping region (marked with white lines) consists of a displacement node point and four nearby antinode points from the transverse vibrations. The 10.1 μm green fluorescent polystyrene particles were levitated to a 3D trapping node above a displacement node point in the microfluidic chamber. Close to the substrate, the trajectories of the 1 μm red fluorescent polystyrene particles indicate that the streaming flows come from the edges of the square-like region (DATVs) and stream towards the center. There, the flow rises up and rotates back toward the edges. Combining the effects of the acoustic radiation force and acoustic streaming induced by two dimensional (2D) SSAWs, it was demonstrated that a 3D trapping node can be generated in the volumetric space above this cubic region within a microfluidic chamber.

In order to achieve 3D manipulation, there is a need to precisely move the trapping nodes along the X, Y, and Z directions. It was demonstrated that the vertical (Z direction) movement of the trapped particles can be achieved by tuning the input power on the IDTs. To move trapped particles in the horizontal plane (X-Y plane), a phase shift strategy was employed: changing the relative phase angle lag (Δϕ) of the RF signals applied to each pair of IDTs can move the DNTVs on the substrate as well as the PNs in the fluid. The change in distance of the DNs (ΔD) along the X or Y direction is given as ΔD=(λ/4π)Δϕ. As a result, the trapped particles in the PNs are moved the same distance along the same direction. For example, at a relative phase angle lag of π/2, the trapped particles move over a distance of π/8. With 3D acoustic tweezers, the randomly distributed 10.1 μm diameter polystyrene particles were first pushed and levitated to 3D trapping nodes in a dot-array configuration. Gradually increasing the relative phase angle lag of the RF signal from 0 to 3π/2 along the Y axis, while keeping all other parameters fixed, it was found that all the trapped particles moved along the Y direction while in the dot-array formation. By decreasing the input power of the RF signals to zero, all the trapped particles settled to the bottom, but kept the dot array formation. The entire process was recorded and is shown in FIG. 5C. Focusing and defocusing indicates that the 3D acoustic tweezers are capable of manipulating micro-particles along the Z direction. The manipulation of particles along the X direction can be achieved in a similar way. With the wide tuning range of the relative-phase-angle lag, one can translocate micro-objects to any desired location within the entire microfluidic chamber and maintain any desired formation of the trapping array, which results in a significant improvement in manipulation dexterity, compared to previous work (14). By demonstrating a vertical range from the substrate surface to the ceiling of the microfluidic chamber, this approach can manipulate micro-particles within the entire volume of a microfluidic chamber. 3D acoustic tweezers provide new possibilities for massively parallel and multi-axis manipulation.

Cell Printing with 3D Acoustic Tweezers

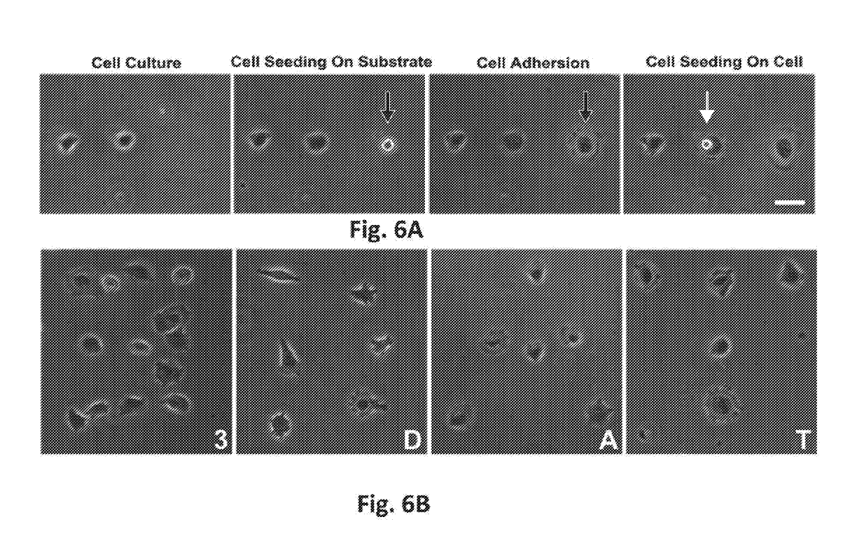

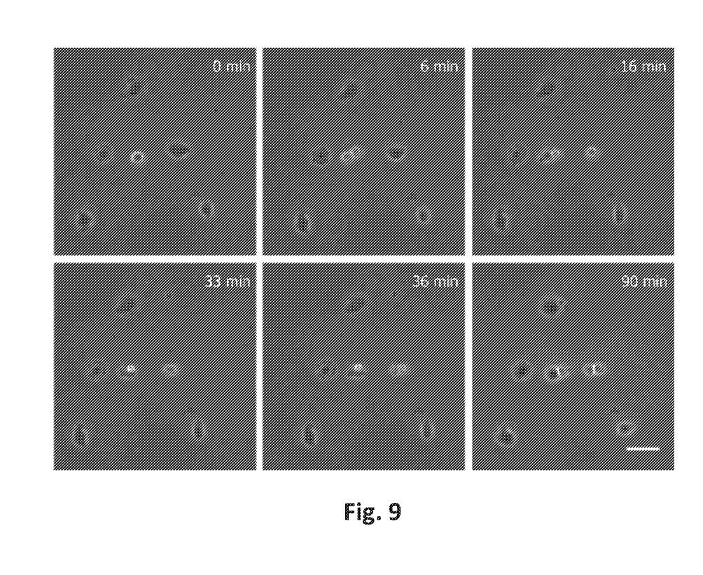

In order to explore potential practical applications of this technology, 3D printing of living cells onto a substrate with customized cell patterns using 3D acoustic tweezers was performed. Earlier work (14, 15) demonstrated that the use of acoustic tweezers had no discernible effect on cell viability, functionality, and gene expression (14, 15). When an acoustic field was applied, living single cells were captured into the 3D trapping nodes. Then, cells were transported along the X or Y directions in a horizontal plane by tuning the phase angle of each IDT pair accordingly. After the delivery of the cells above the target locations, the trapped cells were lowered onto the substrate, or onto other cells that were already in place, by tuning the input power of the acoustic field. As a result, the cells were printed with precise control of the cell number, spacing, and configuration. To demonstrate 3D printing of live cells, a single suspended 3T3 mouse fibroblast was captured and transported to a desired location on the substrate after injecting a cell suspension into the microfluidic chamber (FIG. 6A, indicated with red arrow). Following placement via 3D acoustic tweezers, the cells started to adhere and then spread on to the substrate. In this way, a linear cell array was created by depositing cells one by one (FIG. 6A). One could also position another 3T3 cell on top of the previously adhered cells by employing the precise control of 3D acoustic tweezers (FIG. 6A, indicated with blue arrow) to form a 3D cell assembly. To further demonstrate the capabilities of the 3D acoustic tweezers, this technology was applied to print living cells into complicated configurations, such as numbers and letters. Through single-cell seeding of HeLa S3 cells using acoustic control, cells were printed into the following patterns: “3” “D” “A” “T”, as an acronym for “Three-dimensional Acoustic Tweezers” (FIG. 6B). Cells were able to adhere to the surface and then spread along the surface (FIG. 6B). It was also demonstrated that the adhered cells were able to split and proliferate in the prescribed morphological patterns (FIG. 9). Hence, it has been demonstrated that a versatile acoustic cell printing technique that is capable of single cell resolution and has the ability to manipulate multiple cell types without affecting cell viability.

In this work, it was demonstrated three-dimensional manipulation of micro-particles and biological cells in a microfluidic chamber with two-dimensional SSAWs. Through modeling and experiment, the mechanism behind 3D trapping with acoustic tweezers was investigated. It was shown that 3D trapping originates from the interactions between induced acoustic fields and acoustic streaming in a microfluidic chamber via acoustic vibrations of the substrate. The transverse vibrations of SSAWs induce longitudinal waves into the liquid and establish a Gor'kov potential distribution within the microfluidic chamber. Both the longitudinal and transverse vibrations of SSAWs contribute to the acoustic streaming in the microfluidic chamber; this differs from Rayleigh streaming associated with bulk acoustic waves which are only induced by longitudinal vibrations. The vibration amplitude regulates the transverse, out-of-plane position of the trapped objects by rebalancing the acoustic radiation force, the Stokes drag force, the gravitational force, and the buoyancy force. The predictions of the model are validated by the experimental results. This model accounts for the spatial distribution of the Gor'kov potential and acoustic streaming, and rationalizes the dependence of acoustic manipulation on vibration parameters. 3D trapping node arrays in the microfluidic chamber were created by superimposing two orthogonal displacement nodes and antinodes. These 3D trapping nodes were then translated horizontally or vertically by tuning the location of the displacement nodes or vibration amplitudes. Further demonstrated was three-dimensional manipulation by lifting and pushing micro-particles into 3D trapping node arrays, translating them horizontally, and finally allowing them to sink to the surface. Using an exemplary IDT design, 3D trapping nodes were created and manipulated in a massively parallel formation. However, the independent operation of a 3D node could be realized with dynamic regulation of the vibrations on the substrate through IDT design and RF signal control. Since it uses gentle acoustic vibrations, this method offers several advantages such as dexterous 3D manipulation, digitally programmable and potentially automatable operation, as well as contactless and label-free handling of cells. Finally, can all be done in a simple, low-cost device without any moving parts.

With the exemplary setup and experimental conditions used, one can manipulate a single cell or particle and place it at a desired location down to 1 μm accuracy in the X-Y plane, and 2 μm accuracy in the Z-direction. Since the acoustic wavelength and input power are both instantaneously tunable during experiments, the spatial accuracy (along X, Y or Z direction) of cell/particle placement is in principle only limited by the optical or imaging resolution of the experimental setup. The cell or particle transport time achieved in the present study was in the range of several seconds to a couple of minutes, depending on both the moving velocity and the distance between the cells/particles starting location and the target location. A 10 μm particle or a single cell was able to be moved with an average speed of ˜2.5 μm/s.

Using these 3D acoustic tweezers, the printing of living cells with acoustic manipulation was verified. It was demonstrated that single cells could be picked up, delivered to desired locations, and allowed to adhere and spread on the surface or on top of previously deposited cells (FIG. 6A). Cells were printed into prescribed adherent patterns by acoustic-based single cell seeding. 3D acoustic tweezers were used to pattern cells with control over the number of cells, cell spacing, and the confined geometry, which may offer a unique way to print neuron cells to create artificial neural networks for applications in neuron science and regenerative neuron medicine. For example, during peripheral nerve repair, Schwann cells are naturally aligned into structures, “bands of Büngner”, which guide regenerating neurons to reconnect to their peripheral targets (21). The methods provided herein may offer a unique way to control the spatial distribution of Schwann cells for optimal nerve regeneration. With spatial resolution control down to a single cell, this technology shows the potential to enable layer-by-layer positioning of living cells to create 3D tissue-like structures. This technology could provide new pathways for 3D bio-printing as it addresses the central challenge of replicating tissue structures or even fabricating artificial organs that are made up of multiple cell types and complex geometries, but with single cell control resolution (4). In addition, it is expected that this technology to rebuild the three-dimensional architecture of a tumor, which will aid in the investigation of heterogeneous genetic alterations during tumor growth and metastasis process (22). The ability to precisely transport single cells along three axes using 3D acoustic tweezers may facilitate investigations of a number of challenging problems in biology, particularly those involved in the spatial regulation of cells in 2D or 3D environments (23).

In addition to bio-printing, The 3D acoustic tweezers can aid in the imaging and analysis of biological specimens. Taking a confocal microscope as an example, implementation of the device can provide precise transportation of a target cell or small tissue to the focal area of the microscopic lens. Then the vertical position of the target object can be moved with respect to the focal plane in order to generate a confocal image (24). In addition, the rotation of stable trapped objects with the acoustic streaming patterns discussed above has been demonstrated, which enables the reconstruction of 3D cells or small tissue images. Furthermore, these 3D acoustic tweezers could serve as building blocks in future integrated imaging and analysis systems including microscopes or fluorescent-activated cell sorter (FACS).

### Materials and Methods

### Theoretical Model.

The perturbation theory was used to model the acoustic fields and second-order acoustic streaming in the microfluidic channel, in order to assess two main forces acting on the particles: acoustic radiation force and Stokes drag force. With the perturbation theory, continuity and Navier-Stokes equations are asymptotically expanded into two sets of equations in different order based on a small perturbation. The solution to the first-order equations, together with given boundary conditions that contain SSAWs vibration and partial acoustic radiative losses at the PDMS/fluid interface, yields the acoustic fields. These lead to the determination of the Gor'kov potential and the acoustic radiation force, whereas the solution to the second-order equations identify acoustic streaming. The forces on micro-particles are evaluated based on the two solutions. Detailed formulation, model description, force analysis, and parametric assessment can be found below in Theoretical framework and model setup.

### Theoretical Framework and Model Setup.

The acoustic radiation force and the hydrodynamic drag force induced by nonlinear acoustic streaming are the main forces driving the movement of micro-particles in this system. In order to consider these nonlinear effects, a perturbation approximation was used to analyze the aforementioned forces associated with the acoustic fields and the resulting steady acoustic streaming patterns induced in the fluid. The basic assumption is that the perturbations induced by these acoustic vibration amplitudes should be much smaller than the characteristic length scale of the fluid domain, which also means that the vibrating velocity of the surface due to acoustic waves is much smaller than the acoustic phase velocity. For a surface acoustic wave, its amplitude is typically in the range of 0.1-10 nm, whereas the length scale of SAW-driven microfluidic devices is on the order of 10-1000 μm. This allows us to use the perturbation approximation to numerically investigate the motion of micro-particles driven by standing surface acoustic waves (SSAWs).

The general governing equations for a fluid are the continuity and the Navier-Stokes equations, respectively,

$$\begin{matrix}\frac{\partial ρ}{\partial t} + ∇ \cdot ( ρ v ) = 0 , & ( S1a ) \\ ρ \frac{\partial ρ v}{\partial t} = - ∇ p - ρ ( v \cdot ∇ ) v + μ {∇}^{2} v + ( \frac{1}{3} μ + {μ}_{B} ) ∇ ( ∇ \cdot v ) , & ( S1b )\end{matrix}$$

where ρ, v, p, μ, and μ_{B} are the density, velocity, pressure, dynamic viscosity, and bulk viscosity, respectively. By linearizing the Eq. (S1) using a perturbation approximation, one can obtain the first-order equations for an acoustic field and the time-averaged second-order equations for steady acoustic streaming. The detailed derivation of the two sets of equations can be found in (13). Here, the resulting first-order equations are given directly.

$$\begin{matrix}\frac{\partial {ρ}_{1}}{\partial t} + {ρ}_{0} ∇ \cdot ( {v}_{1} ) = 0 , & ( S2a ) \\ {ρ}_{0} \frac{\partial {v}_{1}}{\partial t} = - {c}_{0}^{2} ∇ {ρ}_{1} + μ {∇}^{2} {v}_{1} + ( \frac{1}{3} μ + {μ}_{B} ) ∇ ( ∇ \cdot {v}_{1} ) . & ( S2b )\end{matrix}$$

The parameters with subscript 1 denote the first-order terms, while the subscript 0 indicates constant properties of a quiescent fluid. c₀ is the speed of sound in the fluid, and it is related to the first-order pressure p₁ with a density ρ₁ through the equation of state p₁=c₀²ρ₁. Substituting the equation of state into Eq. (S2), the first-order equations can be transformed into linear wave equations with viscous attenuation. p₁ and v₁ are the acoustic pressure and the velocity of the acoustic particles, respectively, which give rise to the acoustic radiation force on the particles within the streaming fluid. Assuming a single harmonic time dependence of e^{iωt} on all these fields, the time dependent Eq. (S2) can be transformed into the frequency domain, which is much more efficient for numerical simulations than directly using the time dependent Eq. (S2). Substituting harmonic time-dependent terms into Eq. (S2), one arrives at

iωρ₁+ρ₀∇·v₁=0 (S3a)

iωρ₁v₁=−c₀²∇ρ₁+μ∇²v₁+(⅓μ+μ_{B})∇(∇·v₁) (S3b)

where i is an imaginary term, and co is the angular velocity of acoustic vibrations. The physical fields are the real part of the complex fields in Eq. (S3). For instance, [v₁]=Re(v₁e^{iωt}), where [V₁]on the left side is the physical field, and the v₁ on the right side is the complex field obtained from Eq. (S3).

The second-order time-averaged equations are given as

$$\begin{matrix} \frac{\partial {ρ}_{2}}{\partial t} + {ρ}_{0} ∇ \cdot {v}_{2} + ∇ \cdot ( {ρ}_{1} {v}_{1} ) = 0 , & ( S4a ) \\ {ρ}_{0} \frac{\partial {v}_{2}}{\partial t} + {ρ}_{0} \frac{\partial {v}_{1}}{\partial t} = {ρ}_{0 } ( {v}_{1} \cdot ∇ ) {v}_{1} = - ∇ {p}_{2} + μ {∇}^{2} {v}_{2} + ( \frac{1}{3} μ + {μ}_{B} ) ∇ ( ∇ \cdot {v}_{2} ) . & ( S4b )\end{matrix}$$

Similarly, the parameters with subscript 2 denote the second-order terms. By time-averaging both sides of Eq. (S4), the time-dependent second-order terms on the left hand side disappear and the time-averaged second-order equations, after rearrangements, are

ρ₀∇· [symbol] v₂ [symbol] =−∇· [symbol] ρ₁v₁ [symbol] (S5a)

−∇p₂+μ∇²(v₂)+(⅓μ+μ_{B})∇(∇·v₂)=ρ₀(∂₁v₁)+ρ₀ [symbol] (v₁·∇)v₁ [symbol] · (S5b)

The angled brackets <●> denote a time average over an oscillation period. It can be seen from Eq. (S5) that the equations are actually the continuity and the Navier-Stokes equations with source terms, which drive the flow fields of v₂ and p₂. The source terms, such as the mass source term (−∇·<ρ₁v₁>) in Eq. (S5a) and the force source term (ρ₀<∂v₁/∂t>+ρ₀<(v₁·∇)v₁>) in Eq. (S5b), are products of first-order terms. To solve Eq. (S5), the first-order equations of Eq. (S4) should be solved first. Physically, the non-zero velocity <v₂> is the acoustic streaming velocity.

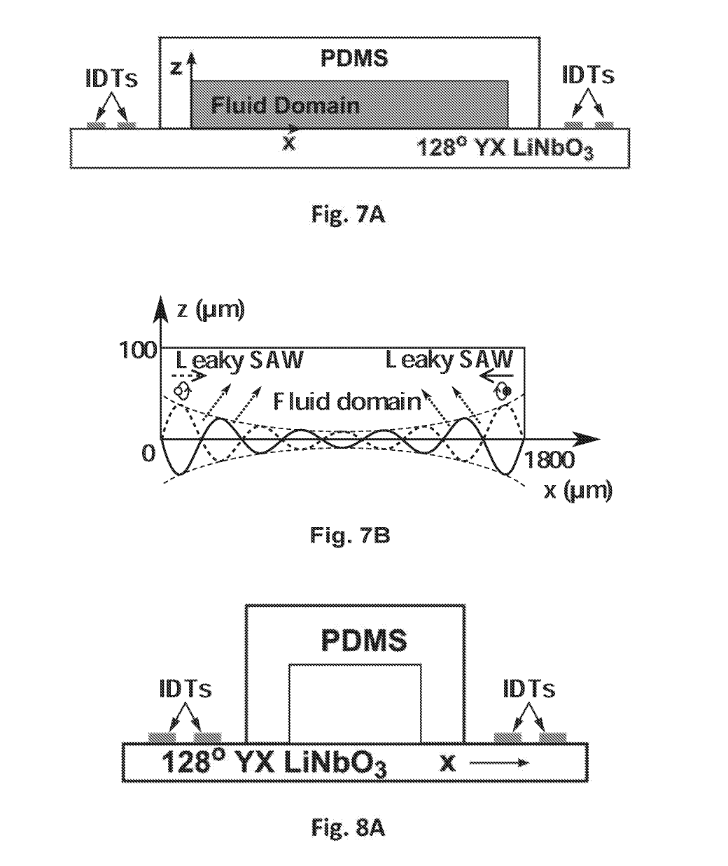

Applying a perturbation analysis, a 2D model was used to analyze the motion of micro-particles in a PDMS chamber actuated by a 1D SSAW. The device used in the experiments is composed of a 128° YX LiNO₃ substrate with two pairs of orthogonally positioned IDTs on the substrate surface. The PDMS chamber is aligned to be parallel to the IDTs and is bonded on the substrate. In order to simplify the experimental system and the associated theoretical model for SSAW-based acoustophoresis in the PDMS chamber, only one pair of parallel IDTs were employed to actuate the fluidic domain within the PDMS chamber. When RF signals are applied to the IDTs, the excited surface acoustic waves (SAWs) travel towards the channel as plane waves. This means that the wave-fronts of the SAWs are parallel to the IDTs and are nearly uniform in the direction parallel to the IDTs. Therefore, a 2D model that approximates the phenomena, in a cross-section (as shown in FIG. 7A) perpendicular to the IDTs, can be used to simplify the verification analysis of the motion of micro-particles in such a fluidic device. Since the dimensions of the PDMS chamber walls are much larger than the acoustic wave amplitudes, and the wave absorption and damping within PDMS layer is strong, its effect on the acoustic fields in the fluidic domain can be modelled as a lossy-wall boundary condition (20), which describes the partial radiative acoustic losses at the liquid/PDMS boundaries, in order to further simplify the model. Conversely, the effects of the motion of the fluid on the surface vibrations generated by the piezoelectric device are neglected. Instead, a commonly imposed leaky SAWs (LSAWs) boundary condition is used to model the actuation of the SSAW at the interface between the substrate and the fluid. The final simplified 2D model is illustrated in FIG. 7B. Only the fluidic domain is numerically solved. The dimensions of the PDMS chamber bonded on the device is 1800 μm×1800 μm×100 μm (length×width×height), and the size of the 2D modelled fluidic domain is 1800 μm×100 μm (width×height).

The first-order acoustic fields are determined by Eq. (S3) and the aforementioned boundary conditions. For the LSAWs boundary at the substrate, the surface particles move in ellipses and the motion is retrograde, that is, the in-plane motion of the particles is counterclockwise when the LSAW propagates from left to right and vice versa. This type of motion means that the vibration of the LSAW consists of both longitudinal and transverse vibrations, and these two motions are separated by a phase lag of 3π/2. Meanwhile, the LSAW decays as it propagates along the interface between the substrate and the fluid domain due to the continuous energy radiation into the fluid. Therefore, by assuming a harmonic time dependence (e^{iωt}) for the LSAW vibrations, the vibrating velocities of the bottom boundary of the fluidic domain, when two LSAWs propagate in opposite directions, can be modelled as

vₓ=εAω{e^{i(ωt-kx)-ωt}+e^{i[ωt-k(x}^{₀}^{-x)]-α(x}^{₀}^{-x)}} (S6a)

v_{y}=−iAω{e^{i(ωt-kx)-αx}−e^{i [ωt-k(x}^{₀}^{-x)]-α(x}^{₀}^{-x)}} (S6b)

where ε is the amplitude ratio between the longitudinal and transverse vibrations; A is the amplitude of the transverse vibration; a is the decay coefficient of the amplitude along x; k is the wave number of the LSAW; and x₀ is the width of the fluidic domain. The ratio ε can be determined from analytical solutions of the Rayleigh wave displacement in the longitudinal (x) and transverse (z) directions, given as²

$$\begin{matrix}{U}_{R} = {A}_{0} {k}_{R} ( {e}^{- {q}_{R} z} - \frac{2 {q}_{R} {s}_{R}}{{k}_{R}^{2} + {s}_{R}^{2}} {e}^{- {s}_{R} z} ) sin ( {k}_{R} x - ω t ) , & ( S7a ) \\ {W}_{R} = {A}_{0} {q}_{R} ( {e}^{- {q}_{R} z} - \frac{2 {k}_{R}^{2}}{{k}_{R}^{2} + {s}_{R}^{2}} {e}^{- {s}_{R} z} ) cos ( {k}_{R} x - ω t ) , & ( S7b )\end{matrix}$$

where A₀ is a constant; k_{R} is the wave number of Rayleigh wave. In Eq. (S7) q_{R} and s_{R} are given by q_{R}=\sqrt{k_{R}²−kₗ²}, and s_{R}=\sqrt{k_{R}²−kₜ²}, respectively, where kₗ and kₜ are the wave numbers for the longitudinal and transverse modes, respectively. According to the parameters given in Table 1, the amplitude ratio ε, i.e., the ratio between amplitude of U_{R} to W_{R}, is 0.7428 in the model. For the decay coefficient α, it is induced by the acoustic energy continuously being attenuated by the fluid, and can be obtained from a dispersion relation for LSAWs, given as (18)

$$4 {k}^{* 2} qs - ( {k}^{* 2} + {s}^{2} ) - i \frac{{ρ}_{0}}{{ρ}_{s}} \cdot \frac{{qk}_{t}^{4}}{\sqrt{{k}_{0}^{2} - {k}^{* 2}}} = 0 , \qquad ( S8 )$$

where k* is the complex wave number of the LSAW; ρₛ is density of the substrate; k₀ is the wave number of the longitudinal wave in the fluid; i is the imaginary unit; q=\sqrt{k²−kₗ²} and s=\sqrt{k²−kₜ²}. By using the relationships between wave numbers and acoustic phase velocities, Eq. (S8) can be transformed into an equation which is independent of the frequency, yielding (25)

$${( 2 - \frac{{c}_{L}^{* 2}}{{c}_{t}^{2}} )}^{2} - \sqrt[4]{1 - \frac{{c}_{L}^{* 2}}{{c}_{l}^{2}}} \sqrt{1 - \frac{{c}_{L}^{* 2}}{{c}_{t}^{2}}} + i \frac{{ρ}_{f} {c}_{L}^{* 2}}{{ρ}_{s} {c}_{t}^{2}} \sqrt{\frac{1 - {c}_{L}^{* 2} / {c}_{l}^{2}}{{c}_{L}^{* 2} / {c}_{f}^{2} - 1}} = 0 , \qquad ( S9 )$$

where cₗ, cₜ, and c_{f} are the acoustic phase velocities of the longitudinal and transverse modes in the 128° YX LiNO₃ substrate, and in the fluid, respectively. CZ in Eq. (S9) is the complex acoustic phase velocity of the LSAW. Using the parameters listed in Table 1, Eq. (S9) has the solution c_{L}*=3900−32.7i (m·s⁻¹). The corresponding wave number is k*=20940+176i (m⁻¹). Physically, the imaginary part of k* is the coefficient of decay for the LSAW. Thus, α in Eq. (S6) is calculated as 176 m⁻¹ in the model.

The other three boundaries in contact with the walls of the PDMS are modeled as lossy-walls in order to account for the acoustic reflection and transmission at the interface between the fluid and the PDMS. This is expressed as (20)

$$n \cdot ∇ {p}_{1} = i \frac{ω {ρ}_{0}}{{ρ}_{w} {c}_{w}} {p}_{1} , \qquad ( S10 )$$

where n is the normal vector with respect to the boundary; ρ_{w} and c_{w} are the density and speed of sound of the PDMS channel material, respectively.

For a spherical particle suspended in an acoustic wave-actuated fluid, the acoustic radiation force, the Stokes drag force induced by acoustic streaming, the gravitational force, and the buoyancy force all need to be taken under consideration. In the model, the first two forces can be obtained from solving the first-order and the second-order equations with the aforementioned boundary conditions.

The acoustic radiation force on an incompressible spherical particle, with a diameter that is smaller than the wavelength of sound can be evaluated using the expression given by Gor'kov (19),

$$\begin{matrix}{F}_{rad} = - ∇ \{ {V}_{p} [ \frac{{f}_{1}}{2 {ρ}_{0} {c}^{2}} 〈 {p}_{1}^{2} 〉 - \frac{3 {ρ}_{0} {f}_{2}}{4} 〈 {v}_{1} \cdot {v}_{1} 〉 ] \} , & ( S11 ) \\ with & \\ {f}_{1} = 1 - \frac{{ρ}_{0} {c}^{2}}{{ρ}_{p} {c}_{p}^{2}} , & ( S12a ) \\ {f}_{2} = \frac{2 ( {ρ}_{p} - {ρ}_{0} )}{2 {ρ}_{p} + {ρ}_{0}} . & ( S12b )\end{matrix}$$

where Vₚ is the volume of the sphere particle, and ρₚ and cₚ are its density and acoustic phase velocity. Here the p₁ and v₁ in Eq. (S11) are the real part of the complex fields in Eq. (S3).

Normally, the Stokes drag force is given by

F_{d}=6πμR( [symbol] v₂ [symbol] −v) (S13)

where R is the radius of the particle, and v is the particle's velocity. Particularly, when a particle moves perpendicularly toward a wall, the Stokes drag formula Eq. (S13) needs to be corrected by an analytical factor to take into account the wall effect. The Stokes drag force with a wall-effect-correction factor is (26, 27)

$$\begin{matrix} {F}_{d} = 6 π μ R ( 〈 {v}_{2} 〉 - v ) χ , & ( S14a ) \\ χ = \frac{4}{3} sinh φ {\sum }_{n = 1}^{∞} \frac{n ( n + 1 )}{( 2 n - 1 ) ( 2 n + 3 )} [ \frac{2 sinh ( 2 n + 1 ) φ + ( 2 n + 1 ) sinh 2 φ}{4 {sinh}^{2} ( n + 1 / 2 ) φ - {( 2 n + 1 )}^{2} {sinh}^{2} φ} - 1 ] , & ( S14b ) \\ φ = {cosh}^{- 1} ( \frac{H}{R} ) , & ( S14c )\end{matrix}$$

where χ is the wall-effect-correction factor, and H is the distance from the bottom wall to the center of the particle. By combining these forces, one can get the dynamic motion equation for the microparticle, which can be expressed as

$$m \frac{dv}{dt} = {F}_{rad} + {F}_{d} + {F}_{b} + {F}_{g} , \qquad ( S15 )$$

where m, F_{b}, and F_{g} are the mass of the particle, the buoyance force, and the gravitational force acting on the particle, respectively.

An FEM-based software package, COMSOL version 4.4 (COMSOL Inc., Burlington, Mass.), was used to solve the abovementioned 2D boundary value problem. The numerical procedure is similar to the one described in (28). First, the first-order Eq. (S4), together with the boundary conditions of Eq. (S6) and Eq. (S10), are solved in the frequency domain by the predefined thermoacoustics solver module in COMSOL. Then, the second-order Eq. (S5) is solved by the laminar flow solver module within COMSOL, which is modified to add a mass source term to the right side of Eq. (S5a) and a body force term to the right side of Eq. (S5b) from the products of first-order acoustic fields. The acoustic radiation force and the Stokes drag force acting on a spherical particle can be determined by inputting the corresponding solutions from the calculations of the second order equations into Eq. (S11) and Eq. (S14). A mesh-independence test has also been done. To confirm the accuracy of the numerical solution, the grid near the bottom boundary was finely meshed, and the distance between nodes in this mesh is much smaller than the thickness of the so-called Stokes-layer or the shear viscosity decay layer, which has a thickness of ∂={square root over (2μl)ρ₀ω))}. The parameters employed in these calculation are summarized in Table 1.

TABLE 1
Parameters used in the numerical simulations.

| | | |
|---|---|---|
| Water | | |
| Density | ρ₀ | 997 kg · m⁻³ |
| Speed of sound | c₀ | 1495 m · s⁻¹ |
| Viscosity | μ | 1 × 10⁻³ Pa · s |
| Bulk viscosity | μ_{B} | 2.4 × 10⁻³ Pa · s |
| Ethanol | | |
| Density | ρ₀ | 789 kg · m⁻³ |
| Speed of sound | c₀ | 1144 m · s⁻¹ |
| Viscosity | μ | 1.082 × 10⁻³ Pa · s |
| Bulk viscosity | μ_{B} | 1.4 × 10⁻³ Pa · s |
| Polystyrene | | |
| Density | ρₚ | 1050 kg · m⁻³ |
| Speed of sound | cₚ | 2350 m · s⁻¹ |
| 128° YX lithium niobate | | |
| Density | ρₛ | 4650 kg · m⁻³ |
| Longitudinal wave speed | cₗ | 7158 m · s⁻¹ |
| Transverse wave speed | cₜ | 4260 m · s⁻¹ |
| Phase velocity of SAW | c_{R} | 3990 m · s⁻¹ |
| Phase velocity of LSAW | c_{L} | 3900 m · s⁻¹ |

### Device Fabrication and Experiment Setup

A procedure for device fabrication is described in (14, 15). Details of the experimental setup and the operating procedures are provided below in Experimental setup and Calibration of vibration amplitude.

### Experimental Setup.

The acoustic device was mounted either on a motorized stage or inside the cell culture chamber (INUBTFP-WSKM-GM2000A, Prior Scientific, Rockland, Mass.) on an inverted microscope (TE2000U, Nikon, Tokyo, Japan). The particle or cell solution was manually injected into the acoustic tweezers device. The images and the videos of the particles or cells were acquired with a 10× microscope objective and a charge-coupled device (CCD) camera (CoolSNAP HQ2, Photometrics, Tucson, Ariz.) or a high speed camera (SA4, Photron, Japan) connected to a computer. Two controllable AC signals generated by a double channel function generator (AFG3102C, Tektronix, Beaverton, Oreg.) and amplified by two amplifiers (25A100A, Amplifier Research, San Diego, Calif.) were connected to one pair of IDTs. The other pair of orthogonally arranged IDTs were powered by a similar setup.

### Calibration of Vibration Amplitude.

In order to correlate the experimental results with the theoretical predictions, the vibration amplitude of the LSAW (A in Eq. (S6)) at different input powers has to be determined. The vibration amplitude was characterized using two different methods.

Method 1. The vibration amplitude was calibrated in the fluidic chamber as a function of the input power by fitting experimental data of acoustic radiation force (ARF) and acoustic streaming measured (FIG. 2D and FIG. 3D), and comparing with the ones calculated by the theoretical model at certain vibration amplitudes. Since the input power P˜A² and F_{rad}˜A², theoretically, the acoustic radiation force F_{rad} is linearly dependent on the input power P. Thus, a linear line through the origin can be used to fit the experimental data of the acoustic radiation force, as shown in FIG. 2D. To calibrate the vibration amplitude, this fitted line is considered to be the magnitude of the acoustic radiation force at different input powers, and was used as reference to compare with the one calculated by the theoretical model until finding the equal one at certain vibration amplitude which was considered as the vibration amplitude at that input power. The calibrated vibration amplitude curve is shown in FIG. 8B, matching with the theoretical prediction that A˜\sqrt{P}. After calibrating the vibration amplitudes, the values were again inputted into the theoretical model to find the acoustic streaming magnitude at different input powers. The theoretically predicted linear line is shown in FIG. 3D which matches with the theoretical prediction. This method accounts the attenuation effects of the PDMS channel wall and the liquid in the microfluidic chamber. In the 1D SSAW microfluidic device, acoustic waves are propagating along the X axis of a 128° YX cut lithium niobate substrate. They travel through the PDMS chamber wall and then encounter the liquid in the microfluidic chamber (FIG. 8A).

Method 2. The vibration amplitude was directly measured on a bare SAW substrate using an optical method. FIG. 8C illustrates the 1D SAW substrate for directly amplitude measurement with an optical vibrometer system (MSA-500 Micro System Analyzer, Polytec Ltd, Hopkinton, Mass.). An additional 4 mm×4 mm gold film with a thickness of 200 nm was deposited in the middle of the IDT pair (along the X axis of a 128° YX cut lithium niobate substrate) to reflect right during the optical measurement. FIG. 8D show the measurement of the longitudinal vibration under different input powers, which are within the same order of magnitude as the aforementioned calculated results.

Further, an analysis was performed with the result using method 1, which is closer to the actual experimental conditions than that from method 2. The amplitude given by method 1 (FIG. 8B) is smaller than the value obtained using method 2 (FIG. 8D). This is because method 1 considers the losses due to the PDMS channel walls and the attenuation effect of the liquid in the microfluidic chamber.

### Data Analysis

The velocity of the micro-particles was measured using Nikon NIS Elements Advanced Research (AR) software (Nikon Inc., Melville, N.Y., USA). A detailed description of the analysis and calculation steps is below.

For the acoustic radiation force analyzed in the experiments, all the 10.1 μm polystyrene particles were suspended in ethanol and were pushed towards pressure nodes by the acoustic radiation force. During this process, the induced Stokes drag force counteracts with this acoustic radiation force so that the acoustic radiation force can be determined by examining the velocity of the particles. The movements of 10.1 μm micro-particles under different input powers were recorded in individual videos with a frame rate of 500/second. The velocity of each single particle can be determined using Nikon NIS Elements Advanced Research (AR) software (Nikon Inc., Melville, N.Y., USA). To better compare with the theoretical predictions, the function f(x)=Vₘₐₓsin(x-x₁)+f₀ was used to fit the moving speed data of each microparticle based on the fact that the acoustic radiation force has a wave form distribution in the horizontal direction. Through the fitting, the Vₘₐₓ of each microparicle, which corresponds to the peak of acoustic radiation force along the trajectory, can be found. Then, the peak of acoustic radiation force can be calculated using F_{rad}=6πRμVₘₐₓ, where R is the radius of the particles, μ is the dynamic viscosity of the medium, and v is the flow velocity relative to the particle. Following this, the acoustic radiation force acting on the micropaticle under different input powers was evaluated. The data was used to match with the corresponding peak horizontal acoustic radiation force in the theoretical model. The detailed parameters used are available in Table 1.

The acoustic streaming velocity was analyzed as the following. After applying SSAW, 1.3 μm micro-particles suspended in ethanol were flowed into the paralleled distributed vortex patterns in the microfluidic chamber, which indicates the induced acoustic streaming. Using the same method, the movements of these small particles (within the X-Y plane, x˜915-960 μm@z˜1-10 μm) under different input powers were recorded in individual videos, and the velocity of each single particle was processed with respect to different input powers.

On-Chip Cell Culture.

Cell culture in the acoustic tweezers device was conducted with a customized cell-culture chamber. Detailed procedures for handling and culturing cells are described below in Cell preparation and culture.

Cell Preparation and Culture.

All the cell lines were purchased from ATCC. 3T3 (CRL-1658) cells were cultured in Dulbecco's Modified Eagles Medium (DMEM, ATCC) supplemented with 10% (vol/vol) FBS and a 1% penicillin-streptomycin solution. HEK 293T (CRL-3216) cells were cultured using Earle's Minimum Essential Medium (EMEM) (Corning, Cellgro) supplemented with 10% (vol/vol) FBS and a 1% penicillin-streptomycin solution. Both adherent cell lines were kept in T-25 cell culture flasks and subcultured twice per week. HeLa S3 cells (CCL-2.2) were cultured in a medium (F-12K medium, ATCC) supplemented with 10% FBS and a 1% penicillin-streptomycin solution. The cells were kept in this suspension within shaker flasks (VWR, mini-shaker) at 100 rpm and sub-cultured twice per week. All the cells are cultured in a 37° C. cell culture incubator with a 5% CO₂ atmosphere. Before the on-chip experiments, cells were harvested, and re-suspended into fresh medium at the desired concentration (0.8-1.2×10⁶ cells/mL). After seeding cells into the device (coating with 2% fibronectin overnight), cells were cultured inside the customized cell-culture chamber. This chamber can provide a stable 37° C. and 5% CO₂ atmosphere for cell culture. In addition, the humid environment necessary for cell culture was maintained by the evaporation of DI water inside the sealed chamber.

### REFERENCES

1.) Bao G, Suresh S (2003) Cell and molecular mechanics of biological materials. Nat

Mater 2(11):715-725.
2.) Berthiaume F, Maguire T J, Yarmush M L (2011) Tissue Engineering and Regenerative Medicine: History, Progress, and Challenges. Annu Rev Chem Biomol Eng 2:403-430.
3.) Wang J Y, et al. (2009) Microfluidics: A new cosset for neurobiology. Lab Chip 9(5):644-652.
4.) Murphy SV, Atala A (2014) 3D bioprinting of tissues and organs. Nat Biotechnol 32(8):773-785.
5.) Guo F, et al. (2013) Probing cell-cell communication with microfluidic devices. Lab Chip 13(16):3152-3162.
6.) Zhang H, Liu K K (2008) Optical tweezers for single cells. J R Soc Interface 5(24):671-690.
7.) Pan Y, Du X W, Zhao F, Xu B (2012) Magnetic nanoparticles for the manipulation of proteins and cells. Chem Soc Rev 41(7):2912-2942.
8.) Albrecht D R, Underhill G H, Wassermann T B, Sah R L, Bhatia S N (2006) Probing the role of multicellular organization in three-dimensional microenvironments. Nat Methods 3(5):369-375.
9.) Calvert P (2007) Printing cells. Science 318(5848):208-209.
10.) Chen C S, Mrksich M, Huang S, Whitesides G M, Ingber D E (1997) Geometric control of cell life and death. Science 276(5317):1425-1428.
11.) Zhang K, Chou C K, Xia X F, Hung M C, Qin L D (2014) Block-Cell-Printing for live single-cell printing. Proc Natl Acad Sci U S A 111(8):2948-2953.
12.) Ding X Y, et al. (2013) Surface acoustic wave microfluidics. Lab Chip 13(18):3626-3649.
13.) Friend J, Yeo L Y (2011) Microscale acoustofluidics: Microfluidics driven via acoustics and ultrasonics. Rev Mod Phys 83(2):647-704.
14.) Guo F, et al. (2015) Controlling cell-cell interactions using surface acoustic waves. Proc Natl Acad Sci U S A 112(1):43-48.
15.) Li P, et al. (2015) Acoustic separation of circulating tumor cells. Proc Natl Acad Sci U S A 112(16):4970-4975.
16.) Gesellchen F, Bernassau A L, Dejardin T, Cumming D R S, Riehle M O (2014) Cell patterning with a heptagon acoustic tweezer—application in neurite guidance. Lab Chip 14(13):2266-2275.
17.) Bourquin Y, et al. (2014) Rare-Cell Enrichment by a Rapid, Label-Free, Ultrasonic Isopycnic Technique for Medical Diagnostics. Angew Chemie Int Ed 53(22):5587-5590.
18.) Viktorov I A (1967) Rayleigh and Lamb Waves: Physical Theory and Applications (Springer, N.Y.).
19.) Gor'kov L P (1962) On the Forces Agting on a Small Particle in an Acoustic Field in an Ideal Fluid. Sov Phys Dokl 6:773-775.
20.) Bruus H (2012) Acoustofluidics 2: Perturbation theory and ultrasound resonance modes. Lab Chip 12(1):20-28.
21.) Svennigsen A F, Dahlin L B (2013) Repair of the Peripheral Nerve: Remyelination that Works. Brain Sciences 3(3):1182-1197.
22.) Waclaw B, et al. (2015) A spatial model predicts that dispersal and cell turnover limit intratumour heterogeneity. Nature 525(7568):261-264.
23.) Manz B N, Groves J T (2010) Spatial organization and signal transduction at intercellular junctions. Nat Rev Mol Cell Biol 11(5):342-352.
24.) McDermott G, Le Gros M A, Knoechel C G, Uchida M, Larabell C A (2009) Soft X-ray tomography and cryogenic light microscopy: the cool combination in cellular imaging. Trends Cell Biol 19(11):587-595.
25.) Vanneste J, Buhler O (2011) Streaming by leaky surface acoustic waves. Proc R Soc A Math Phys Eng Sci 467(2130):1779-1800.
26.) Brenner H (1961) The Slow Motion of a Sphere through a Viscous Fluid towards a Plane Surface. Chem Eng Sci 16(3-4):242-251.
27.) Ryu S, Matsudaira P (2010) Unsteady Motion, Finite Reynolds Numbers, and Wall

Effect on Vorticella convallaria Contribute Contraction Force Greater than the Stokes Drag. Biophys J 98(11):2574-2581.
28.) Muller P B, Barnkob R, Jensen M J H, Bruus H (2012) A numerical study of microparticle acoustophoresis driven by acoustic radiation forces and streaming-induced drag forces. Lab Chip 12(22):4617-4627.

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one or all of the group members are present in, employed in or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of manipulating one or more particles in a reservoir in three dimensions, wherein the reservoir is disposed on a substrate, comprising:
generating a first surface acoustic wave (SAW) and a second SAW along a first axis of the substrate, wherein the first SAW and the second SAW are generated from opposite sides of the reservoir;
generating a third SAW and a fourth SAW along a second axis of the substrate, wherein the third SAW and the fourth SAW are generated from opposite sides of the reservoir, and wherein the first axis and the second axis intersect in the reservoir;
manipulating the one or more particles in the reservoir to move along the first axis and/or the second axis by varying a frequency and/or a phase of at least one of the first, the second, the third, and/or the fourth SAW; and
manipulating the one or more particles in the reservoir to move along an axis that is orthogonal to the first axis and the second axis by varying an acoustic power of at least one of the first, the second, the third, and/or the fourth SAW.

2-3. (canceled)

4. The method of claim 1, wherein the first axis is at an angle ranging from 1-90 degrees relative to the second axis.

5-6. (canceled)

7. The method of claim 1, wherein the first acoustic wave, the second acoustic wave, the third acoustic wave, and the fourth acoustic wave are generated by a first, a second, a third, and a fourth, interdigital transducer (IDT).

8. The method of claim 1, wherein the first acoustic wave, the second acoustic wave, the third acoustic wave, and the fourth acoustic wave are generated by a first, a second, a third, and a fourth segmented interdigital transducer (S-IDT).

9-10. (canceled)

11. The method of claim 1, wherein the one or more particles comprise one or more organic particles, inorganic particles, biological cells, or microorganisms.

12-16. (canceled)

17. The method of claim 1, wherein the frequency of any one of the first, the second, the third, and/or the fourth surface acoustic waves is increased.

18-30. (canceled)

31. The method of claim 1, wherein the reservoir comprises one or more trapping nodes.

32-35. (canceled)

36. The method of claim 1, further comprising imaging one or more of the one or more particles.

37. (canceled)

38. An device for manipulating one or more particles in three dimensions, comprising:
a reservoir on a substrate;
a first pair of surface acoustic wave (SAW) generators, having a first variable power/frequency SAW generator that vibrates the substrate in response to a first input signal and a second variable power/frequency SAW generator that vibrates the substrate in response to a second input signal, wherein the first and second variable power/frequency SAW generators are disposed on the substrate and on opposing sides of the reservoir to generate surface acoustic waves within the reservoir having a first SAW path;
a second pair of surface acoustic wave (SAW) generators, having a third variable power/frequency SAW generator that vibrates the substrate in response to a third input signal and a fourth variable power/frequency SAW generator that vibrates the substrate in response to a fourth input signal, wherein the third and fourth variable power/frequency SAW generators are disposed on the substrate and on opposing sides of the reservoir to generate surface acoustic waves within the reservoir having a second SAW path;
wherein the first SAW path and the second SAW path are different; and
wherein the first, second, third and fourth input signals have an input power, a frequency and a phase.

39. The device of claim 38, further comprising an electronic control circuit providing the first, second, third, and fourth input signals to the first, second, third, and fourth variable power/frequency SAW generators, wherein the electronic control circuit is configured to independently vary the input power, the frequency, and the phase of each of the first, second, third and fourth input signals.

40-46. (canceled)

47. The device of claim 38, wherein the reservoir comprises a second substrate.

48-54. (canceled)

55. The device of claim 38, wherein the SAW generator is an interdigital transducer (IDT).

56. The device of claim 38, wherein the SAW generator is a segmented interdigital transducer (S-IDT).

57-62. (canceled)

63. The device of claim 38, wherein the reservoir comprises at least one inlet and at least one outlet.

64-65. (canceled)

66. The device of claim 38, wherein the first SAW path is disposed at an angle ranging from 1-90 degrees relative to the second SAW path.

67-71. (canceled)

72. A method of manipulating one or more particles in three dimensions using tunable surface acoustic waves, the method comprising:
introducing a fluid suspension comprising one or more particles to the reservoir of the device of claim 38, wherein the first SAW path denotes a first axis, the second SAW path denotes a second axis and a path orthogonal to the first and second SAW paths denotes a third axis;
generating surface acoustic waves in the fluid suspension from the first and second pair of SAW generators;
manipulating the one or more particles to move along the first axis and/or the second axis by adjusting the frequency and/or phase of one or more of the first, second, third and/or fourth input signals; and
manipulating the one or more particles to move along the third axis by adjusting the input power of one or more of the first, second, third and/or fourth input signals.

73-86. (canceled)

87. A method of printing one or more biological cells onto a substrate, comprising
providing a reservoir disposed on a substrate, wherein the reservoir comprises one or more biological cells;
generating a first surface acoustic wave (SAW) and a second SAW along a first axis of the substrate, wherein the first SAW and the second SAW are generated from opposite sides of the reservoir;
generating a third SAW and a fourth SAW along a second axis of the substrate, wherein the third SAW and the fourth SAW are generated from opposite sides of the reservoir, and wherein the first axis and the second axis intersect in the reservoir;
manipulating the one or more biological cells in the reservoir to move along the first axis and/or the second axis by varying a frequency and/or a phase of at least one of the first, the second, the third, and/or the fourth SAW; and
manipulating the one or more biological cells in the reservoir to move along an axis that is orthogonal to the first axis and the second axis by varying an acoustic power of at least one of the first, the second, the third, and/or the fourth SAW; and
depositing the one or more cells onto a cell substrate.

88. (canceled)

89. The method of claim 87, wherein the reservoir comprises a second substrate.

90-93. (canceled)

94. The method of claim 87, wherein the cell substrate comprises collagen, fibronectin, an RGD peptide, an extracellular matrix (ECM) protein, or a growth factor.

95. The method of claim 87, wherein one or more cells are deposited onto the cell substrate within 1 uM of a target site on the cell substrate or the second substrate.

96. (canceled)
